(19) United States
(12) Patent Application Publication
(10) Pub. No.: US 2026/0269024 A1
(43) Pub. Date: Sep. 10, 2026

(54) ACCELERATED PHASE SPACE EXPLORATION OF MOLECULAR SYSTEMS USING NEURAL NETWORKS

(71) Applicant: NEC Laboratories Europe GmbH, Heidelberg (DE)

(72) Inventors: Henrik CHRISTIANSEN, Heidelberg (DE); Federico ERRICA, Heidelberg (DE); Francesco ALESIANI, Heidelberg (DE)

(21) Appl. No.: 19/167,880

(22) PCT Filed: Nov. 20, 2023
(86) PCT No.: PCT/IB2023/061688

§ 371 Date: Sep. 23, 2025

Related U.S. Application Data
(60) Provisional application No. 63/530,265, filed on Aug. 2, 2023.

(51) Int. Cl. G16C 20/70; G06F 30/27

(52) U.S. Cl. CPC G16C 20/70; G06F 30/27

(57) ABSTRACT

A computer-implemented method for accelerating the simulation of a molecular system is provided. The method includes learning a first set of parameters by executing a Hamiltonian Monte Carlo method on the molecular system based on a set of initial conditions. A simulation of the molecular system is executed based on the first set of parameters. Thermodynamics expectation values of the molecular system are predicted based on the executed simulation. The predicted thermodynamics expectation values are provided for a downstream machine learning (ML) task. The method has applications including, but not limited to use cases in artificial intelligence (AI), drug development, medical diagnostics/applications, healthcare, material design catalyst design and high performance computing, to optimize predictions, improve model (e.g., neural network) performance or support decision making.

### CROSS-REFERENCE TO PRIOR APPLICATION

This application is a U.S. National Phase application under 35 U.S.C. § 371 of International Application No. PCT/IB2023/061688, filed on Nov. 20, 2023, and claims benefit to U.S. Provisional Application No. 63/530,265, filed on Aug. 2, 2023, the entire contents of which is hereby incorporated by reference herein. The International Application was published in English on Feb. 6, 2025 as WO 2025/027381 A1 under PCT Article 21(2).

### FIELD

The present invention relates to artificial intelligence (AI) and machine learning (ML), and, in particular to a method, system and computer-readable medium and computer program product for accelerated phase space exploration of molecular systems using neural networks, with applications including, but not limited to drug discovery, polymer modelling and new material discovery for catalyst.

### BACKGROUND

Conventional methods to study properties of a molecular system are costly and time consuming, in terms of computational time, power, and resources available which prohibits many researchers from utilizing such methods.

### SUMMARY

In an embodiment, the present invention provides a computer-implemented method for accelerating the simulation of a molecular system. The method includes learning a first set of parameters by executing a Hamiltonian Monte Carlo method on the molecular system based on a set of initial conditions. A simulation of the molecular system is executed based on the first set of parameters. Thermodynamics expectation values of the molecular system are predicted based on the executed simulation. The predicted thermodynamics expectation values are provided for a downstream machine learning (ML) task. The method has applications including, but not limited to, use cases in artificial intelligence (AI), drug development, medical diagnostics/applications, healthcare, material design, catalyst design and high performance computing, to optimize predictions, improve model (e.g., neural network) performance or support decision making.

### BRIEF DESCRIPTION OF THE DRAWINGS

Embodiments of the present invention will be described in even greater detail below based on the exemplary figures. The present invention is not limited to the exemplary embodiments. All features described and/or illustrated herein can be used alone or combined in different combinations in embodiments of the present invention. The features and advantages of various embodiments of the present invention will become apparent by reading the following detailed description with reference to the attached drawings which illustrate the following:

FIG. 1 schematically illustrates a Hamiltonian Monte Carlo system, in accordance with embodiments of the present invention;

FIG. 2 schematically illustrates an alternation of molecular dynamics and Monte Carlo steps in the bottom portion of FIG. 1, in accordance with embodiments of the present invention;

FIG. 3 illustrates an integration step of the Hamiltonian Monte Carlo system of FIG. 1 implemented by the molecular dynamics integration component, in accordance with embodiments of the present invention;

FIG. 4 schematically illustrates the input and output of an example application of an Hamiltonian Monte Carlo simulation according to an embodiment of the present invention to the problem of protein to protein property prediction, in accordance with embodiments of the present invention;

FIG. 5 schematically illustrates an example application of an Hamiltonian Monte Carlo simulation according to an embodiment of the present invention for the simulation of the properties of a polymer, in accordance with embodiments of the present invention;

FIG. 6 schematically illustrates an example application of an Hamiltonian Monte Carlo simulation according to an embodiment of the present invention for the simulation of the properties of small molecules for catalyst design or small drug molecule development, in accordance with embodiments of the present invention;

FIG. 7 is a block diagram of an exemplary processing system, which can be configured to perform any and all operations disclosed herein, in accordance with embodiments of the present invention;

FIG. 8 graphically shows experimental results of Loss L of Alanine-Dipeptide in dependence of the time step Δt, in accordance with embodiments of the present invention;

FIG. 9 graphically shows experimental results of Loss L as a function of training epochs for an initial value of Δt of (a) 1.0, (b) 1.5, and (c) 2.0, in accordance with embodiments of the present invention;

FIG. 10 graphically shows autocorrelation results, in accordance with embodiments of the present invention;

FIG. 11 shows example trajectories of the one-dimensional harmonic oscillator as obtained from the HMC, in accordance with embodiments of the present invention;

FIG. 12 graphically shows experimental results of acceptance p and logarithm of squared jump as a function of time step and number of integration steps for the one-dimensional oscillator, in accordance with embodiments of the present invention;

FIG. 13 graphically shows experimental results of Loss L_{N} as a function of Δt and n for no jittering (s=0) and with jittering (s=¼), the logarithm of the autocorrelation time extracted from the time series of the potential energy, how the loss is rescaled with the computational effort Lₙ/n, and the logarithm of the autocorrelation time in units of the computational effort nτ, in accordance with embodiments of the present invention;

FIG. 14 graphically shows experimental results of loss surface as a function of Δt and n, in accordance with embodiments of the present invention;

FIG. 15 graphically shows alanine dipeptide, in accordance with embodiments of the present invention;

FIG. 16 graphically shows experimental results of influence of parameter b on the loss shown for alanine dipeptide for b=2, b=3, and b=4 along with the corresponding logarithm of the autocorrelation time of the potential energy, in accordance with embodiments of the present invention

FIG. 17 graphically shows experimental results of Loss L per computational effort Lₙ/n as a function of Δt and n, the value of Δt as a function of the learning epoch t for the three initial conditions, the corresponding loss for three initial conditions as a function of training epoch t, and the weights c as a function of the corresponding time step n for different training epochs t, in accordance with embodiments of the present invention; and

FIG. 18 depicts graphs that show loss L as a function of epoch t for different initial values, the individual Δtᵢ per atom for the initial value of Δt=0.9 fs, the final learned value of Δtᵢ after training is shown for the different initial values of Δt, and the mean value of integration steps n̅ as a function of learning epoch t, in accordance with embodiments of the present invention; and

FIG. 19 depicts a violin plot for the distribution of the time step for the four different atom types present in alanine dipeptide, in accordance with embodiments of the present invention.

### DETAILED DESCRIPTION

Embodiments of the present invention provide a method, system and computer-readable medium and computer program product that accelerates the phase space exploration of molecular systems with the help of neural networks. The classical leap-frog integration step of molecular dynamics (MD) is augmented by learned neural networks, which allow to optimize the simulation condition and accelerate the exploration of the free energy landscape. Special care is taken to allow use of this for molecules. The method provides an approximate two-fold acceleration of phase space exploration measured in terms of integration steps.

Controlled experiments of a molecular system to study its properties is a costly and time-consuming activity, both in terms of computational time, power and resources and in terms of physical and expert resources. Numerical simulation is the de-facto alternative, but computational methods pose various challenges. To properly measure properties of the underlying physical system, the technical problem of how to develop numerical simulators that behave according to the basic assumptions of the system under study must be overcome. One standard approach is to describe the system in terms of forces acting at the atomistic level and simulate the system over time by sampling form the Boltzmann distribution. Therefore, for the accurate estimation of the thermodynamic properties of physical molecular systems, the technical problem of how to sample from the correct Boltzmann distribution (at fixed temperature) must be overcome.

The sampled free-energy surface resulting from sampling from the Boltzmann distribution, however, has many local minima (it is rugged) and the local dynamics are very slow (i.e., small time step necessary in molecular dynamics requiring in the order of millions of time steps of simulation), and it is difficult to construct efficient update proposals for Monte Carlo. An additional technical problem is the control of external system parameters such as temperature, which in a molecular dynamics setting is not solved in a unified way.

Embodiments of the present invention provide to overcome these technical problems to accelerate the exploration of phase space by learning a rescaling of the velocities and forces in the (microcanonical) integration steps employed in the setting of Hamiltonian Monte Carlo in such a way that less samples are needed to achieve the same accuracy. This is achieved by introducing strategies that avoid/remove the learning of output that only leads to translational/rotational movements of the molecule.

According to a first aspect, the present disclosure provides a computer-implemented method for accelerating the simulation of a molecular system. The method includes learning a first set of parameters by executing a Hamiltonian Monte Carlo method on the molecular system based on a set of initial conditions. A simulation of the molecular system is executed based on the first set of parameters. Thermodynamics expectation values of the molecular system are predicted based on the executed simulation. The predicted thermodynamics expectation values are provided for a downstream machine learning task.

According to a second aspect, the method according to the first aspect further comprises the Hamiltonian Monte Carlo method using a leap-frog integrator including learnable functions realized by neural networks.

According to a third aspect, the method according to the first or the second aspect further comprises the neural networks being configured such that a computed loss is invariant to translations and rotations of molecules of the molecular system.

According to a fourth aspect, the method according any of the first to the third aspect further comprises the neural networks being configured such that outputs of the neural network transform equivariantly to rotations of molecules of the molecular system.

According to a fifth aspect, the method according any of to first to the fourth aspect further comprises the neural networks being configured to learn atom dependent time steps so as to only learn parameters that cannot lead to translations or rotations.

According to a sixth aspect, the method according to any of the first to the fifth aspect further comprises that learning the first set of parameters includes only considering rescaling parameters that are invariant to a molecular transformation.

According to a seventh aspect, the method according to the first to the sixth aspect further comprises that the set of initial conditions comprises a description of the molecular system, functional form of forces and energy, and a second set of parameters for the simulation.

According to an eighth aspect, the method according to the first to the seventh aspect further comprises that the description of the molecular system comprises a list of molecules to be investigated, and physical conditions of the molecular system.

According to a ninth aspect, the method according to the first to the eighth aspect further comprises that the simulation of the molecular system is executed for a fixed number of compute operations.

According to a tenth aspect, the method according to the first to the ninth aspect further comprises that the simulation of the molecular system is executed for a fixed time.

According to an eleventh aspect, the method according to the first to the tenth aspect further comprises that the predicted thermodynamics expectation values comprise binding affinities for ligand-protein or protein-protein pairs, and the downstream machine learning task simulates ligand-protein or protein-protein interaction for drug discovery in medical AI/healthcare.

According to a twelfth aspect, the method according to the first to the eleventh aspect further comprises that the predicted thermodynamics expectation values comprise material properties such as stability, responses to shear, or responses to solvents, and the downstream machine learning task simulates bio-plastic or plastic thermodynamical and mechanical properties.

According to a thirteenth aspect, the method according to the first to the twelfth aspect further comprises that the predicted thermodynamics expectation values comprise binding energies as proxy for efficiency of catalysts used in the molecular system, and the downstream machine learning task simulates small molecules for catalysis for energy storage and production.

A fourteenth aspect of the present disclosure provides a computer system programmed for accelerating the simulation of a molecular system, the computer system comprising one or more hardware processors which, alone or in combination, are configured to provide for execution of the following steps: learning a first set of parameters by executing a Hamiltonian Monte Carlo method on the molecular system based on a set of initial conditions; executing a simulation of the molecular system based on the first set of parameters for a fixed number of compute operations; predicting thermodynamics expectation values of the molecular system based on the executed simulation; and providing the predicted thermodynamics expectation values for a downstream machine learning task; or a method according to any of the first to thirteenth aspects.

A fifteenth aspect of the present disclosure provides a tangible, non-transitory computer-readable medium for accelerating the simulation of a molecular system having instructions thereon, which, upon being executed by one or more processors, provides for execution of the following steps: learning a first set of parameters by executing a Hamiltonian Monte Carlo method on the molecular system based on a set of initial conditions; executing a simulation of the molecular system based on the first set of parameters for a fixed number of compute operations; predicting thermodynamics expectation values of the molecular system based on the executed simulation; and providing the predicted thermodynamics expectation values for a downstream machine learning task; or a method according to any of the first to thirteenth aspects.

According to an embodiment, the approach is based on a generic Hamiltonian Monte Carlo, which is composed of a molecular dynamics sub-system and a Monte Carlo sub-system, where:
1. The molecular dynamics sub-system handles the evolution in time.
2. The Monte Carlo sub-system handles the proposal of new initial states for the molecular dynamics sub-system and the acceptance of the final state proposed by molecular dynamics sub-system.

The Hamiltonian Monte Carlo gets as input:
1) The atomic system specification, for example in the form of a simplified molecular-input line-entry (SMILE) format or as coordinates, atomic number and possibly bonds (protein database file format).
2) The functional form of forces and energy, for example in terms of a force field, which is a function of two or more atoms (specified by coordinates and type) and returns the forces acting on each atom and the total energy of the system. Instead of a classical force field, the forces can also be computed using a neural network potential or a direct call to an external program performing density functional theory (differentiable for the problem at hand).
3) Parameters of the simulation, for example the temperature, or other soft or hard constraints, but also parameters of the MD and MC steps, such as for example the number of integration steps for the MD part of HMC.

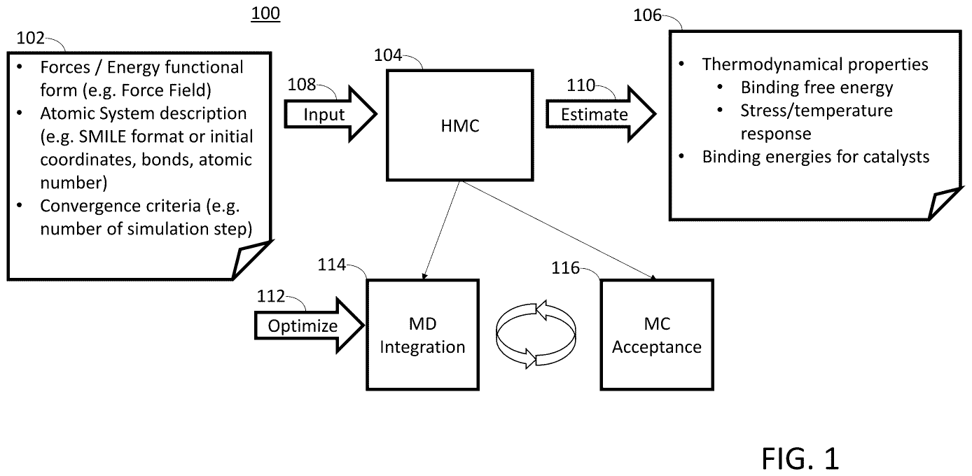

FIG. 1 schematically illustrates a Hamiltonian Monte Carlo system, according to an embodiment of the present invention. System 100 of FIG. 1 includes a Hamiltonian Monte Carlo system 104 that takes an input 108 and provides an output 110. The input fields of the Hamiltonian Monte Carlo system 104 are shown in block 102. For example, the Hamiltonian Monte Carlo system 104 takes as input 108, the description of the atomic system. The description of the atomic system can include simplified molecular entry in the simplified molecular-input line-entry (SMILE) format. The simplified molecular-input line-entry system is a specification in the form of a line notation for describing the structure of chemical species using short ASCII strings. Alternatively, the atomic system description may be provided using initial coordinates of the atoms, bonds that are in play between the atoms and the atomic number of the atoms that are in the atomic system.

The input 108 also includes forces that are in play in the atomic system. For example, the input may include a force field. A classical force field is a function which takes as an input, two or more atoms (specified by coordinates and type) and returns the forces acting on each atom and the total energy of the system. Alternatively, instead of using a classical force field, the forces can also be computed using a neural network potential or a direct call to an external program performing density functional theory (differentiable for the problem at hand).

The input 108 also includes parameters of the simulation. Parameters of the simulation can include, for example, the temperature, or other soft or hard constraints, but also parameters of the molecular dynamics and Monte Carlo steps, such as for example the number of integration steps for the molecular dynamics part of Hamiltonian Monte Carlo. In some embodiments, the parameters can also include convergence criteria which specifies the number of simulation steps in the molecular dynamics and the Monte Carlo simulations.

The Hamiltonian Monte Carlo 104 processes the provided inputs 108 and determines an output 110. In some embodiments, the output 110 includes an estimate. The estimate as shown in block 106 can include thermodynamical properties of the atomic system, such as binding free energy and stress/temperature responses. The output estimate 110 can also include an estimation of the binding energies of various catalysts as shown in block 106.

The Hamiltonian Monte Carlo model 104 uses a combination of the molecular dynamics integration subsystem 114 and the Monte Carlo subsystem 116 to determine the estimate 110. The molecular dynamics subsystem 114 handles the evolution of the atomic system in time, while the Monte Carlo subsystem 116 proposes new states for the molecular dynamics subsystem 116. In response to receiving proposed states from the Monte Carlo subsystem 116, the molecular dynamics integration subsystem 114 provides either an acceptance or a rejection of the proposed states, which is used to train the molecular dynamics subsystem 114 to make better proposals. The training of the molecular dynamics subsystem 114 in this manner happens without the use of training data which allows for the training of molecular dynamics subsystem 114 in cases where systems data may not available. In some embodiments, this method of training also reduces the overhead memory requirements of the Hamiltonian Monte Carlo 104. In some embodiments, the molecular dynamics integration subsystem is also optimized block 112.

The generic Hamiltonian Monte Carlo approach consists of L leap-frog integrator steps followed by a Metropolis-Hastings acceptance barrier. Since the usual leap-frog integrator is volume preserving, the acceptance criterion is essentially given by the ratios of the Boltzmann distribution. For small enough time steps, the acceptance rate is close to 100% (due to total energy conservation), which is clearly not desirable (balance between no movement in space and no acceptance).

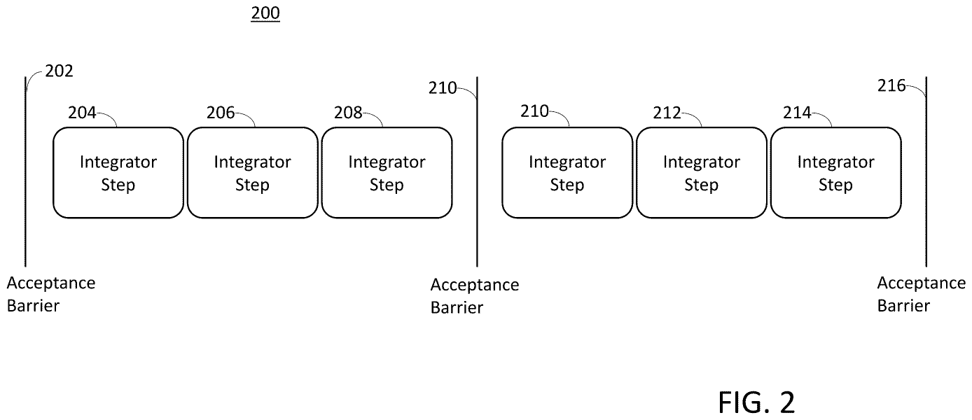

FIG. 2 schematically illustrates an alternation of molecular dynamics (MD) and Monte Carlo (MC) steps in the bottom portion of FIG. 1, according to an embodiment of the present invention. The system 200 of FIG. 2 depicts leap-frog integrator steps 204, 206, and 208 being separated from leap-frog integrator steps 210, 212, and 214 by Metropolis-Hastings acceptance barrier 210. In some embodiments, the leap-frog integrator steps 204, 206, and 208 can be separated by other leap-frog integrator steps using a second Metropolis-Hastings acceptance barrier 202. Similarly, the leap-frog integrator steps 210, 212, and 214 can be separated by other leap-frog integrator steps using a second Metropolis-Hastings acceptance barrier 216. The leap-frog integrator step is a method for numerically integrating differential equations. The Metropolis-Hastings algorithm is a Markov chain Monte Carlo model for obtaining a sequence of random samples from a probability distribution from which direct sampling is difficult. This sequence can be used to approximate the distribution (e.g. to generate a histogram) or to compute an integral (e.g. an expected value).

In some embodiments, an atomic system is described by coordinates in 3-dimensional space. There are interactions in-between the atoms. The molecular dynamics subsystem 114 integrates this motion in such a way that the total energy of the system is conserved. In some embodiments, the positions of the atoms are updated using a leapfrog-integrator. In some embodiments, the leap-frog integrators use a finite time step, and thus the time evolution of the atomic system is not completely computed. The Monte Carlo acceptance/rejection step provides the complete evolution of the atomic system in time. That is, it provides the convergence to the target distribution. The atomic system description provides information about the initial positions, the atom types and the interactions between them, which finally determines the evolution of the system and the expected behavior under external conditions.

It was previously proposed to alter the leap-frog integrator step to incorporate a learned scaling of positions, velocities, forces and a translating contribution (see Levy, Daniel, et al., “Generalizing Hamiltonian Monte Carlo with Neural Networks,” ICLR 2018, arXiv:1711.09628 (Mar. 2, 2018) (hereinafter “Levy”); Liu, Shaofan, et al., “Adversarially Training MCMC with Non-Volume-Preserving Flows,” Entropy 2022, 24(3), 415 (Mar. 16, 2022) (hereinafter “Liu”); John, C. M, “Investigating Machine Learning methods to replace Hybrid Monte Carlo in simulations of Hubbard Model,” Masters Thesis, Rheinische Friedrich-Wilhelms-Universitaet Bonn (2021) (hereinafter “John”); and Hirt, Marcel, et al., “Entropy-based adaptive Hamiltonian Monte Carlo,” NeurIPS 2021, arXiv:2110.14625 (Oct. 27, 2021) (hereinafter “Hirt”), with each of Levy, Liu, John and Hirt being incorporated by reference herein). This is then, in general, not volume preserving, and the determinant of the Jacobian is nonzero (and must be accounted for). A massive speed-up was observed. The rescaling was constructed to allow for a fast evaluation of the determinant of the Jacobian. However, among other technical deficiencies, each of Levy, Liu, John and Hirt cannot be applied to molecules. John merely applies a L2HMC algorithm to a simple lattice model from high-energy physics without any further adjustments, and is not suitable for molecules, and Hirt uses a different loss to square jump distance and is not applied to molecules, and does not address translation/rotational invariance.

In some embodiments of the present disclosure, the neural networks are used with adapted definition of loss or equivariant in the integration step on the fly so that the HMC learns the parameters of the HMC. The output of the neural networks is also used after fixing the parameters.

Other approaches work to accelerate Hamiltonian Monte Carlo by speeding-up the calculation of gradients of the “Hamiltonian”, but do not learn ways to rescale the parameters of Hamiltonian Monte Carlo with the goal to accelerate the phase space exploration (see Thaler, Denny, et al. “Enhanced Hamiltonian Monte Carlo simulations using Hamiltonian neural networks,” Proceedings in Applied Mathematics and Mechanics, vol. 22, issue 1, e202200188, Weinheim, Wiley-VCH (2023) (hereinafter “Thaler”); Li, Lingge, et al., “Neural Network Gradient Hamiltonian Monte Carlo,” Computational Statistics 2019, arXiv:1711.05307 (Oct. 26, 2018) (hereinafter “Li”); and Cobb, Adam D., et al., “Scaling Hamiltonian Monte Carlo Inference for Bayesian Neural Networks with Symmetric Splitting,” Machine Learning, arXiv:2010.06772 (Oct. 14, 2020) (hereinafter “Cobb”), with each of Thaler, Li and Cobb being incorporated by reference herein). These approaches are thus not comparable to the approach according to embodiments of the present invention, which works in another orthogonal direction to accelerate HMC. Nor are these approaches capable of accelerating integration. Chao, Wei-Lun, et al., “Exponential Integration for Hamiltonian Monte Carlo,” Proceedings of the 32^{nd} International Conference on Machine Learning Research, PMLR 37:1142-1151 (2015) (hereinafter “Wei-Lun”), which is hereby incorporated by reference herein, describes to use a different, exponential integrator, and no learned parameters, and is merely based on analytically solving a Gaussian part of integration.

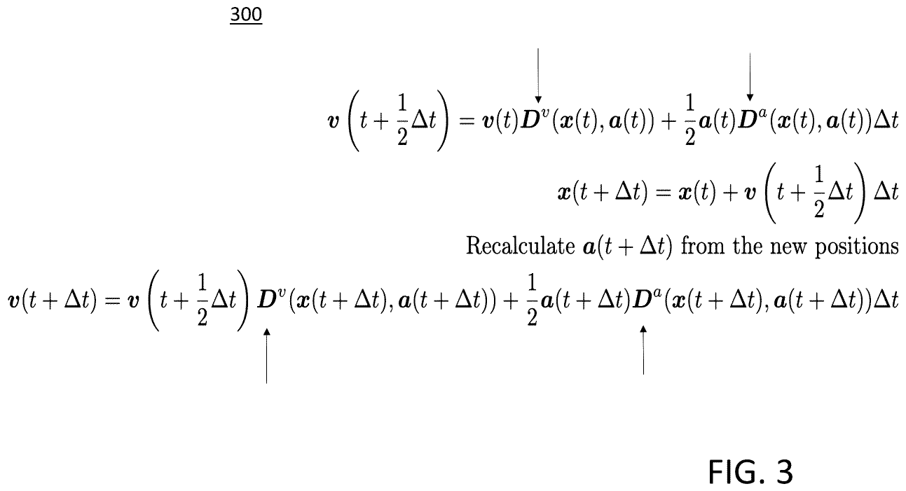

System 300 of FIG. 3 illustrates leap-frog integrator according to an embodiment of the present invention, which is altered by the learnable “functions” D^{v,a}, which are here realized by neural networks. They are marked by arrows in FIG. 3. Additionally, it is possible to learn optimal values of Δt. In some embodiments, single or multiple neural networks may be used to realize the learnable “functions” D^{v,a}. In some embodiments, the leapfrog integrator is extended by the function D^{v,a}, which are not present in a standard formulation of the integrator.

In the equations 300 presented in FIG. 3, x represents positions of the components of the atomic system, v represents the velocity of the components of the atomic system, t represents time, and Δt represents an integration timestep.

In some embodiments, in molecular dynamics simulations, Δt is determined by the fastest oscillating atoms. Thus, Δt is one of the parameters learned. Finding the optimal values is part of the optimization with gradients.

The system can be trained “on the fly”: starting from a configuration of the molecular system to be studied, the approach according to an embodiment of the present invention evolves the system using the learned integrator (with initially randomly initialized functions D^{v,a}) for a certain number of steps, and then it treats the resulting set of configurations as a batch of data samples to train the parameters of the functions D^{v,a}. Hence, embodiments of the present invention advantageously do not require a labeled dataset to train the machine learning part of the integrator. The data generated during training does not need to be discarded, but can be used in conjunction with the data generated after finishing the training of the parameters. Thus, there is only a small overhead associated with learning rescaling parameters of Hamiltonian Monte Carlo. This advantageously enables faster computation and training, and/or conserving computational power and resources.

While this approach is rather general and can be applied to the typical problems considered in statistics, it is not directly applicable for physical simulations of molecules. Embodiments of the present invention extend this approach to molecules, which poses following technical challenge: Absolute (coordinate) translations would decrease the loss (given by the acceptance probability times the movement in phase space), but there would be no actual change of the system. This can be observed when investigating the autocorrelation on physical observables, such as the potential energy.

Since in Markov chain Monte Carlo simulations subsequent samples are correlated, this autocorrelation is to be considered when estimating errors on the thermodynamic expectation values. For this, one estimates the so-called effective sample size, which is reciprocally related to the autocorrelation time.

Embodiments of the present invention provide three solutions to solve the foregoing technical challenge for simulations of molecules:
1. Adapting the definition of the loss to be invariant to translations and rotations (using relative distances of the coordinates or observables, that are invariant to translations and rotations, such as the potential energy or the radius of gyration). This approach can serve as an alternative to generalized ensemble methods such as metadynamics. This way, the learned parameters have no “reason” to learn translational movement.
2. Using equivariant neural networks to learn a rescaling of critical parameters of the leap-frog integrator step. This way, all rescalings transform with the molecule, leading to no net translations. One example of usable equivariant architectures is “Tensor field networks”.
3. Only considering rescaling parameters that are invariant to the molecule's transformation, such as translations and rotations (using a neural network to only predict time step). This may also be formulated in such a way that a parameter does not depend on the positions, which allows for fast execution. For example, one may learn a different time step for every call to the integrator in a sequence of updates before the acceptance barrier.

Embodiments of the present invention can be practically applied to effect further improvements in various technical fields, for example, where use cases can apply simulations of various systems, molecules, chemical structures, materials, entities, etc. for decision making, predictions or classifications. For example, embodiments of the present invention have medical use cases, for example, for AI-assisted drug development (AIDD), development of personalized vaccines, predicting treatments (e.g., to assist doctors or diagnostics), drug and protein interactions, lengths of stays in hospitals. Embodiments of the present invention could also be applied for material design, for example, to predict chemical compositions of materials or catalysts for chemical processes. Embodiments of the present invention are not limited to these use cases, but can be applied to others where Hamiltonian Monte Carlo simulations support decision-making.

In an embodiment, the present invention can be applied for the simulation of protein-protein or protein-ligand binding for drug discovery as a use case. Drugs are always administrated to a living body, that is, under physical conditions that correspond to a living organism. This includes the blood properties (viscosity, density) and temperature. Therefore, to decide whether a given drug/protein interacts strongly with a given protein it is not sufficient to only estimate the purely energetic contributions. It is rather important to accurately sample the events associated with binding and non-binding to estimate the free-energy associated with the binding. This set-up can be used to screen for good candidates before synthesizing in a wet lab. This problem also occurs in the construction of personalized cancer vaccines, where the goal is to trigger an immune-response specific to the cancer cells. The data source for inputs to the simulation are here the amino acid sequence of the target protein as well as the atoms and their bonds for ligand-protein systems and the amino-acid sequence of the second protein for a protein-protein system. For the simulations, special care is taken for the protein-water interactions and for the use of periodic boundary conditions (which are used to approximate a large surrounding water environment). While the system behaves invariantly regarding translation and rotation of the whole system, this is no longer true for the individual components (they interact with each other). This is solved by application of the approach according to embodiments of the present invention. The output can be a binding free-energy as a proxy for the effectiveness of a medical treatment.

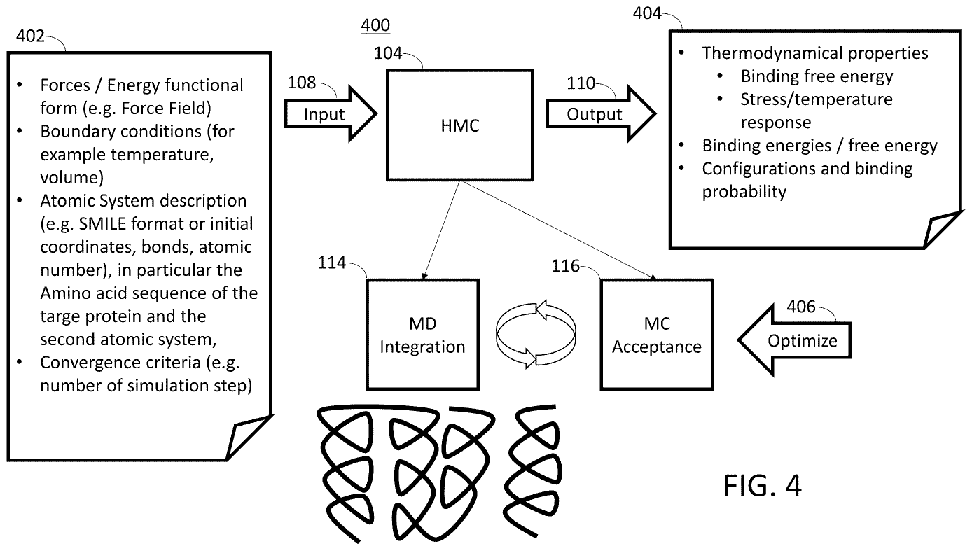

FIG. 4 schematically illustrates the input and output of an example application of an Hamiltonian Monte Carlo simulation according to an embodiment of the present invention to the problem of protein to protein property prediction. System 400 of FIG. 4 is similar to system 100 of FIG. 1, and includes a Hamiltonian Monte Carlo system 104 that takes input 108 and provides an output 110. The Hamiltonian Monte Carlo system 104 uses a combination of the molecular dynamics integration subsystem 114 and the Monte Carlo acceptance subsystem 116 as shown in FIG. 1. Parameters of the molecular dynamics integration subsystem 114 is optimized using block 406. In some embodiments, the parameters of the molecular dynamics subsystem 114 influence the acceptance of the Monte Carlo subsystem 116. The potential inputs 108 of the Hamiltonian Monte Carlo system 104 are listed in block 402. The inputs listed in block 402 include the forces that are in play in the atomic system, the description of the atomic system, and the convergence criteria as described in block 102 of FIG. 1. The description of the atomic system as shown in block 402 of FIG. 4 also includes a particular amino acid sequence and a target protein and a second atomic system. In some embodiments, there are two atomic systems in contact with each other. Both atomic systems are described by proteins that are described by their amino acid sequences. In some embodiments, one of atomic systems is much larger than the other system.

This helps the Hamiltonian Monte Carlo 104 to determine the effectiveness of the amino acid in binding with the target protein. Additionally, the block 402 of FIG. 4 differs from block 102 of FIG. 1 because block 402 also includes boundary conditions, including temperature and volume as inputs 108 for the Hamiltonian Monte Carlo model 104.

As shown in FIG. 1, the Hamiltonian Monte Carlo 104 processes the provided inputs 108 and determines an output 110. In some embodiments, the output 110 includes an estimate. Similar to block 104 of FIG. 1, the estimate outputs shown in block 404 can include thermodynamical properties of the atomic system, such as binding free energy and stress/temperature responses. The output estimate 110 can also include an estimation of the binding energies of various catalysts as shown in block 106. Additionally, the block 404 of FIG. 4 differs from block 104 of FIG. 1 because block 404 also includes configurations and binding probability of the drug with the protein in the body.

In an embodiment, the present invention can be applied for the simulation of molecules, for example, polymer molecules for a bio-plastic and/or a plastic thermodynamic and mechanical property prediction as a use case. Plastics are made up of polymers, which themselves are molecules consisting of often repeating subunits called monomers forming long chains. A single polymer chain can consist of several thousand repeating units. These individual polymer chains interact with each other to form emergent behavior desirable in plastic material. Physically, these systems are extremely hard to simulate since they show signatures of glassy dynamics. The data source for inputs to the simulation include the repeating unit and the density of polymers in the system. Running the simulation enables to estimate the thermodynamic properties. Here, a full sampling of the free-energy is typically not achieved, so that one resorts to more direct measurements, such as investigating perturbations to the system (external stress/temperature change). Application of the approach according to embodiments of the present invention enable accelerating these simulations, allowing for more rapid exploration and the investigation of system sizes, which were hitherto not accessible. As output, the simulations can be used to estimate thermodynamic properties such as stress and temperature response, longevity, and biodegradability. Due to the limited amount of fossil material left, development of bioplastics is an important modern technical field.

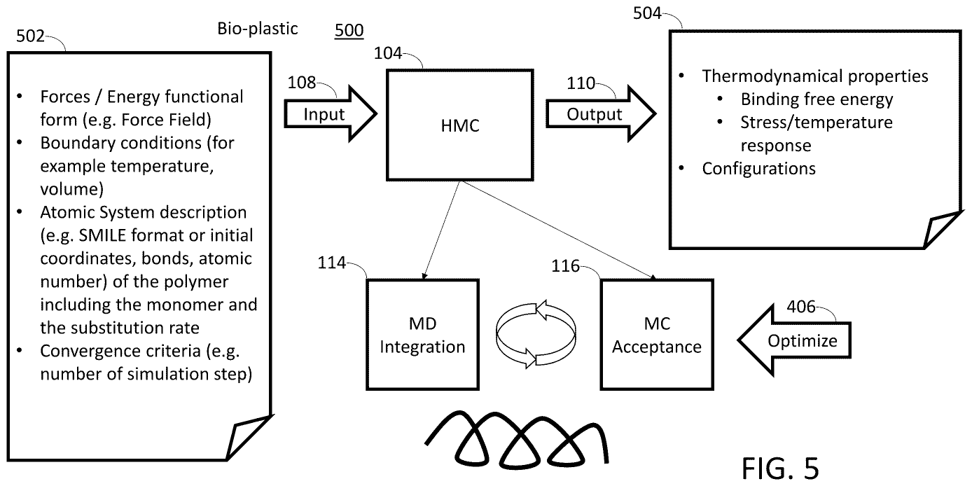

FIG. 5 schematically illustrates an example application of an Hamiltonian Monte Carlo simulation according to an embodiment of the present invention for the simulation of the properties of a polymer. System 500 of FIG. 5 is similar to system 100 of FIG. 1, and includes a Hamiltonian Monte Carlo system 104 that takes input 108 and provides an output 110. The Hamiltonian Monte Carlo system 104 uses a combination of the molecular dynamics integration subsystem 114 and the Monte Carlo acceptance subsystem 116 as shown in FIG. 1. The Monte Carlo subsystem 116 is optimized using block 406. The potential inputs 108 of the Hamiltonian Monte Carlo system 104 are listed in block 502. The inputs listed in block 502 include the forces that are in play in the atomic system, the description of the atomic system, and the convergence criteria as described in block 102 of FIG. 1. The description of the atomic system as shown in block 502 of FIG. 5 also includes a monomer for a specific polymer and the substitution rate of the monomer. Additionally, the block 502 of FIG. 5 differs from block 102 of FIG. 1 because block 502 also includes boundary conditions, including temperature and volume as inputs 108 for the Hamiltonian Monte Carlo model 104.

As shown in FIG. 1, the Hamiltonian Monte Carlo 104 processes the provided inputs 108 and determines an output 110. In some embodiments, the output 110 includes an estimate. Similar to block 104 of FIG. 1, the estimate outputs shown in block 504 can include thermodynamical properties of the atomic system, such as binding free energy and stress/temperature responses. Additionally, the block 504 of FIG. 5 differs from block 104 of FIG. 1 because block 504 also includes configurations of the atomic system as outputs of the Hamiltonian Monte Carlo 104. In some embodiments, the molecular dynamics integration 114 is designed to update the positions of the atomic system, resulting in different configurations. The Monte Carlo acceptance 116 step checks, on a high level, whether the steps follow the target distribution.

In an embodiment, the present invention can be applied for the simulation of small molecules, for example, for catalysis for energy storage and production or mechanization as a use case. For the investigation of the performance of catalysts, the binding energy may be considered as proxy. It is important to account for quantum-mechanical interactions, necessitating the use of methods incorporating these effects. One of the most used approaches is density-functional theory (DFT), which solves the electron density as a proxy for the systems energy. Alternatively, one may learn a “classical” approximation of this using neural networks, often referred to as neural force fields. Both approaches can be plugged into the method according to embodiments of the present invention without additional considerations. The data source for inputs to the simulation can be a surface, described as atom types and crystalline structure and/or several candidates of catalysts, described as atom types. Application of the method according to embodiments of the present invention thus has two advantages in these approaches: Accelerating the overall simulation by learning parameters of the integrator and switching between the quantum mechanical accuracy (used for Monte Carlo acceptance/rejection step) and classical force fields (used in integration step) without losing any information. As output, as a proxy of effectiveness, the binding energy is obtained, which has shown empirical good correlation with the performance of the final catalysts.

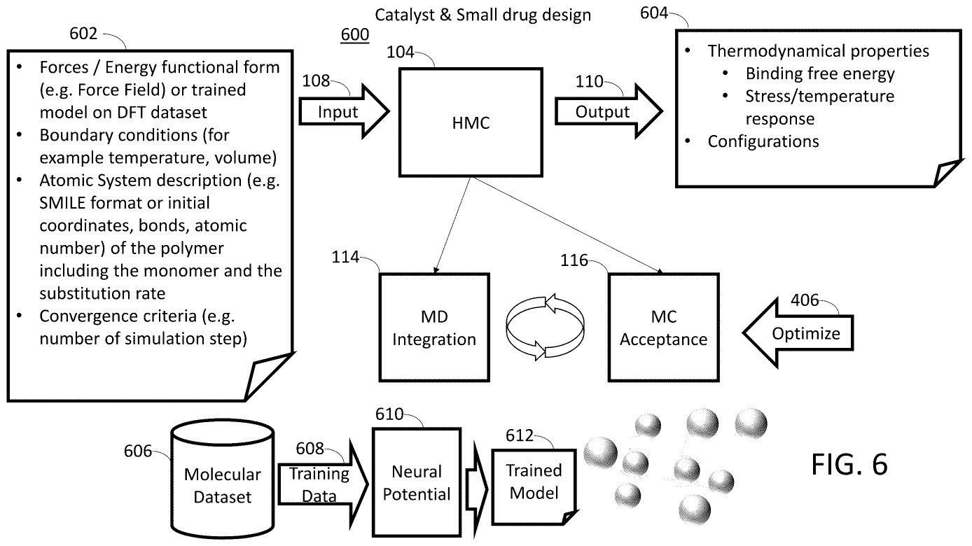

FIG. 6 schematically illustrates an example application of an Hamiltonian Monte Carlo simulation according to an embodiment of the present invention for the simulation of the properties of small molecules for catalyst design or small drug molecule development, according to embodiments of the present invention. System 600 of FIG. 6 is similar to system 100 of FIG. 1, and includes a Hamiltonian Monte Carlo system 104 that takes input 108 and provides an output 110. The Hamiltonian Monte Carlo system 104 uses a combination of the molecular dynamics integration subsystem 114 and the Monte Carlo acceptance subsystem 116 as shown in FIG. 1. The Monte Carlo subsystem 116 is optimized using block 406. The potential inputs 108 of the Hamiltonian Monte Carlo system 104 are listed in block 602. The inputs listed in block 602 include the forces that are in play in the atomic system, the description of the atomic system, and the convergence criteria as described in block 102 of FIG. 1. The description of the atomic system as shown in block 602 of FIG. 6 also includes a particular monomer and the substation rate of the monomer. Additionally, the block 602 of FIG. 6 differs from block 102 of FIG. 1 because block 602 also includes boundary conditions, including temperature and volume as inputs 108 for the Hamiltonian Monte Carlo model 104.

As shown in FIG. 1, the Hamiltonian Monte Carlo 104 processes the provided inputs 108 and determines an output 110. In some embodiments, the output 110 includes an estimate. Similar to block 104 of FIG. 1, the estimate outputs shown in block 604 can include thermodynamical properties of the atomic system, such as binding free energy and stress/temperature responses. Additionally, the block 604 of FIG. 6 differs from block 104 of FIG. 1 because block 604 also includes configurations of the atomic system as outputs of the Hamiltonian Monte Carlo 104.

While in some embodiments, the force field property of the thermodynamic system may be computed using density functional theory, a classical force field determination using neural networks may also be used. System 600 also depicts a classical force field used in the integration step which starts with a molecular dataset 606, training data 608, neural potential 610 and the trained model 612. The molecular dataset 606 includes information regarding molecules and the forces acting on them in a system. A part of this molecular dataset 606 may be used to form a training dataset 608 which is used to along with neural potential 608 to generate a trained model 612 that is able to predict forces acting on atoms in an atomic system. This predictions are used as one of the input 108 to the Hamiltonian Monte Carlo instead of the forces computed by the density functional theory as shown in block 602.

In an embodiment, the present invention provides a method for accelerating the simulation of molecules by learning critical parameters of HMC, the method comprising the steps of:
1) Introducing learned parameters (rescaling) in the integration step of HMC (integration step: FIG. 1 “MD integration”, FIG. 3 with learnable functions D^{v,a}).
   a. The neural networks are constructed so that the loss is invariant to translations and rotations.
   b. Alternatively, construct network architecture in such a way that it produces output that transforms equivariantly to rotations (but invariantly to translations).
   c. It is also provided to only learn parameters that cannot lead to translations or rotations, such as learning a (atom-dependent) time step.
2) Set up the system and simulation by specifying what molecules should be investigated under which conditions (FIG. 1 “Input”)
3) Initially run HMC to learn parameters “on the fly” with optimization of loss as a proxy of phase space exploration or autocorrelation time (FIG. 1 “Optimize” arrow). In some embodiments, “on the fly” learning of the parameters implies that the parameters are not learned earlier and are learned by first running the HMC, then the parameters are fixed and the HMC is run again. Convergence to the steady-state distribution can still be achieved this way. In some other embodiments, the optimization of the loss can be performed using gradient based methods, such as gradient descent or methods based on top of gradient descent, such as Adam or AdaGrad, for example. This is one of the features of using a fully differentiable set-up. The goal of an efficient simulation method is to have as little correlation as possible between subsequent configuration, while still picking them from the target distribution. This is measured in the autocorrelation time, which has an implication on the estimated errors of the observables. The autocorrelation time, however, needs very long to converge. Therefore, a proxy is introduced, which uses only “local” information, i.e., not the full sequence of states. In that sense, the loss is “only” a proxy for the “true” autocorrelation time.
4) Fix all learned parameters and run a production simulation for a chosen amount of time/compute operations. In some embodiments, the HMC is run again once the parameters are fixed for a chosen amount of time/compute operations.
5) Estimate thermodynamics expectation values of the molecular system.
6) Depending on the embodiment or use case, examples of relevant thermodynamic expectation values, for example, can include:
   a. Drug discovery: Binding affinities (binding free-energy differences) for ligand-protein or protein-protein pairs.
   b. Plastics: Material properties, such as stability, responses to shear, or responses to solvents (such as salt-water, acids, etc.).
   c. Catalysts: Calculation of binding energies as a proxy for the efficiency of the catalysts.

Embodiments of the present invention provide for the following improvements over existing technology:
1. By learning a proper rescaling of the atomic velocities and accelerations in the MD integrator balancing phase exploration and acceptance rates through the use of equivariant neural networks, the phase space exploration of molecular systems in HMC simulations is accelerated. This improves computation speed, reduces training time, and enables to improve compute power and conservation of computational resources. Moreover, it improves response time and speeds up processes and ML in the technical fields of application.
2. By guiding learning with a translation and rotation-invariant objective function (such as considering only relative distances or properties of the molecule that do not change under these operations) that enhances phase-space exploration, the ML system can be prevented from exploring degenerate solutions, such as trivial translations and rotations, to accelerate the phase space exploration of molecular systems in HMC simulations. This likewise improves computation speed, reduces training time, and enables to improve compute power and conservation of computational resources.
3. Allows to apply learned HMC to molecular systems, and allows to optimize the HMC automatically without hyper-parameter search of some parameters to accelerate the simulation.
4. Enables the reduction of the time needed for computation of the simulation, which for example can translate to the success of a project based on molecular dynamics that otherwise would not have would have not succeeded, but for this reduction.

Thus, embodiments of the present invention provide an improved HMC system including a trainable HMC pipeline. In particular, embodiments of the present invention enhance the computer systems of existing technology by providing for a learnable HMC system for molecules, for example, learning parameters using a neural network that is equivariant. Not only does this enhance the computational functionality of the HMC systems, but also enables to reduce computation time, autocorrelation times and error estimates on expectation values of existing HMC systems. A prototype has demonstrated the reduced autocorrelation times for molecules which can be achieved with the improved HMC system according to embodiments of the present invention. Roughly, a speed-up of factor two to three has been observed.

Embodiments of the present invention can be used directly in molecular simulation platforms, incorporated into existing simulation software (e.g., OpenMM, Amber, Gromacs, etc.), integrated into the NEC The Wise platform, for drug, vaccine or material discovery, etc.

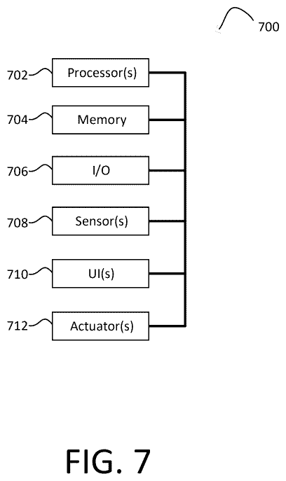

Referring to FIG. 7, a processing system 700 can include one or more processors 702, memory 704, one or more input/output devices 706, one or more sensors 708, one or more user interfaces 710, and one or more actuators 712. Processing system 700 can be representative of each computing system disclosed herein.

Processors 702 can include one or more distinct processors, each having one or more cores. Each of the distinct processors can have the same or different structure. Processors 702 can include one or more central processing units (CPUs), one or more graphics processing units (GPUs), circuitry (e.g., application specific integrated circuits (ASICs)), digital signal processors (DSPs), and the like. Processors 702 can be mounted to a common substrate or to multiple different substrates.

Processors 702 are configured to perform a certain function, method, or operation (e.g., are configured to provide for performance of a function, method, or operation) at least when one of the one or more of the distinct processors is capable of performing operations embodying the function, method, or operation. Processors 702 can perform operations embodying the function, method, or operation by, for example, executing code (e.g., interpreting scripts) stored on memory 704 and/or trafficking data through one or more ASICs. Processors 702, and thus processing system 700, can be configured to perform, automatically, any and all functions, methods, and operations disclosed herein. Therefore, processing system 700 can be configured to implement any of (e.g., all of) the protocols, devices, mechanisms, systems, and methods described herein.

For example, when the present disclosure states that a method or device performs task “X” (or that task “X” is performed), such a statement should be understood to disclose that processing system 700 can be configured to perform task “X”. Processing system 700 is configured to perform a function, method, or operation at least when processors 702 are configured to do the same.

Memory 704 can include volatile memory, non-volatile memory, and any other medium capable of storing data. Each of the volatile memory, non-volatile memory, and any other type of memory can include multiple different memory devices, located at multiple distinct locations and each having a different structure. Memory 704 can include remotely hosted (e.g., cloud) storage.

Examples of memory 704 include a non-transitory computer-readable media such as RAM, ROM, flash memory, EEPROM, any kind of optical storage disk such as a DVD, a Blu-Ray® disc, magnetic storage, holographic storage, a HDD, a SSD, any medium that can be used to store program code in the form of instructions or data structures, and the like. Any and all of the methods, functions, and operations described herein can be fully embodied in the form of tangible and/or non-transitory machine-readable code (e.g., interpretable scripts) saved in memory 704.

Input-output devices 706 can include any component for trafficking data such as ports, antennas (i.e., transceivers), printed conductive paths, and the like. Input-output devices 706 can enable wired communication via USB®, DisplayPort®, HDMI®, Ethernet, and the like. Input-output devices 706 can enable electronic, optical, magnetic, and holographic, communication with suitable memory 706. Input-output devices 706 can enable wireless communication via WiFi®, Bluetooth®, cellular (e.g., LTE®, CDMA®, GSM®, WiMax®, NFC®), GPS, and the like. Input-output devices 706 can include wired and/or wireless communication pathways.

Sensors 708 can capture physical measurements of environment and report the same to processors 702. User interface 710 can include displays, physical buttons, speakers, microphones, keyboards, and the like. Actuators 712 can enable processors 702 to control mechanical forces.

Processing system 700 can be distributed. For example, some components of processing system 700 can reside in a remote hosted network service (e.g., a cloud computing environment) while other components of processing system 700 can reside in a local computing system. Processing system 700 can have a modular design where certain modules include a plurality of the features/functions shown in FIG. 7. For example, I/O modules can include volatile memory and one or more processors. As another example, individual processor modules can include read-only-memory and/or local caches.

The following provides further background and description of exemplary embodiments of the present invention, which may overlap to some extent with some of the information provided above. To the extent the terminology used to describe the exemplary embodiments may differ from the terminology used to describe the above embodiments, a person having skill in the art would understand that certain terms correspond to one another in the different embodiments. Features described in the following can be combined with features described above in various embodiments.

Over the last decades, the simulation of biomolecular system has progressed a lot, dramatically increasing the reachable system size and timespan. Simulations of biomolecular systems are predominantly performed using either molecular dynamics or Monte Carlo simulations. While the former method, in its basic formulation, solves Newton's equations numerically, the latter (directly) samples from the target distribution. Both approaches can be used to sample the canonical ensemble (where a way to alter the velocities in molecular dynamics is needed), i.e., a system in contact with a thermal bath and can, for many purposes, be used as plug-in replacements. A priori, there is thus no apparent advantage of one method over the other, but the devil is in the details.

In molecular dynamics, the time needs to be discretized, and the equation of motions are integrated numerically. This introduces short-time errors as well as accumulating errors, leading for example to a violation of energy conversation (for microcanonical simulations) in the long-time limit. While small enough time steps are important for the correct physical evolution, they lead to severely impeded dynamics since the system only slowly moves through phase-space. Additional complications are introduced when the system is couple to a thermostat; here, the velocities as proxy for the system's temperature are modified. There is no canonical way to do this, manifested by many alternative approaches available in the literature.

In Monte Carlo, on the other hand, there is no error introduced by numerical integration and no adverse effects from controlling the temperature, as this is directly manifested in the acceptance probability. However, trial configurations should be generated in a clever way, i.e., to come up with a move set which constitutes major configuration changes without being penalized by a very low acceptance. There are a couple of well accepted move-sets for different systems, but of course no optimal one. Traditionally, the proposed moves are random, i.e., their proposal probability does not depend on the current configuration in order to guarantee detailed balance.

In the literature, there already exist a couple of approaches to combine both methods, where one of the most studied one is Hamiltonian Monte Carlo, which originally was referred to as hybrid Monte Carlo. The basic idea is to propagate the system using (microcanonical) molecular dynamics simulations, but add an acceptance of proposals obtained in this way using Monte Carlo. In case of rejection of a move, the system is reset (in the typical Monte Carlo fashion) to the previous state. The additional step required for Hamiltonian Monte Carlo is to randomly draw new velocities, as otherwise the resulting configuration would be identical after rejection. Compared to the individual methods on their own, this combined approach has the advantage that there is no adverse effect of the numerical integration or external control of temperature, while providing a systematic way to propose trial configurations. Of course, these trial configurations proposed by this approach have a high acceptance probability, as only the numerical error leads to a rejection (after all, the total energy is in principle conserved in a microcanonical molecular dynamics simulation).

In some embodiments, there is an optimal balance between large movements in phase-space and the acceptance rate. Moves with a lower acceptance probability have the potential to lead to larger changes in the system, effectively leading to a smaller autocorrelation time measured in terms of the number of generated configurations. A way to tune this in Hamiltonian Monte Carlo would be the adaptation of the number of molecular dynamics steps to arrive at a trial configuration and the time step within the integrator. The tuning of the time-step will be the first discussion point, highlighting the problem of multiple-minima in finding optimal values. For this, a fully differentiable molecular dynamics framework build on top of TorchMD will be used. Instead of trying to find optimal values of the parameters of Hamiltonian Monte Carlo, adapting the functional form of the integrator may be attempted. One natural approach is to use neural networks to learn effective ways of how to integrate the equation of motions, where special care has to be taken to fulfill detailed balance. The velocity verlet integrator could be altered and a learned scaling of positions/velocities as well as translational contributions could be introduced. Levy describes a couple of trial distributions with multiple minima, mostly consisting of variants of Gaussian distributions. Additionally, Levy employs a latent-variable generative model to generate images, where it showed superior performance compared to variational auto encoders. This showed that it not only reduced the autocorrelation time significantly, but also allowed the sampling of several (free) energy minima from a single simulation.

The general methodology of the aforementioned method is modified and adapted to the general framework of molecular dynamics simulations and demonstrate its performance for the common test system Alanindipeptide. A short overview over the involved simulation methods is given, followed by an conceptual introduction to the employed machine learning methods. Results for Alanindipeptide are presented, discussing the achieved improvements in simulation speed and an analysis of the learning process and obtained parameters. Finally, a conclusion is provided.

In the following, the goal is to simulate a classical system, which consists of particles/atoms interacting via classical potentials. This system is in coupled to a heatbath, i.e., the target is to simulate in the canonical ensemble. Then, each microstate occurs with a probability that is given by:

$${P}^{e q} ( x ) = \frac{1}{Z} {e}^{- U ( x ) / ( kT )} , \qquad ( 1 )$$

where Z=∫e^{-U(x)/kT} dx is the partition function (in statistic often simple referred to as normalizing constant), U(x) is the (potential) energy of a microstate depending on the atoms positions, k is the Boltzmann constant, and T is the temperature of the heatbath. While the evaluation of the partition function would provide access to many thermodynamic observables, this is in practice not possible since this necessitates a summation over all possible microstates. Instead, approximate expectation values by producing samples from the target distribution is attempted.

In Monte Carlo, producing samples from the target distribution is conceptually, a priori, straightforward. The main idea behind importance sampling Monte Carlo is to build a Markov chain, starting with a random configuration and subsequently progressing by proposing new configurations (which only depend on the current configuration). There is some freedom in choosing the transition probabilities Wᵢⱼ=W (ξᵢ, ξⱼ) between microstate ξᵢ and ξⱼ. In Monte Carlo for biomolecular systems, typically ξ=x, i.e., a set of Cartesian coordinates, but in general this can be any configurational information. One of the most flexible choices for the acceptance criterion between states is the original Metropolis algorithm which reads:

$${w}_{i j} = w ( {ξ}_{i} , {ξ}_{j} ) = min ( 1 , \frac{f ( {ξ}_{j} , {ξ}_{i} )}{f ( {ξ}_{i} , {ξ}_{j} )} \frac{{P}^{e q} ( {ξ}_{j} )}{{P}^{e q} ( {ξ}_{i} )} ) , \qquad ( 2 )$$

where fᵢⱼ=f(ξᵢ, ξⱼ) is the selection probability for a potential update to a new microstate and P^{eq} is the target equilibrium distribution, for the canonical ensemble specified in Eq. (1). Here, the partition function cancels, since one is only interested in the ratio of the equilibrium distributions. This then leads to the transition probability:

$${W}_{i j} = \{ \begin{matrix}{f}_{i j} {w}_{i j} & j \neq i \\ {f}_{i i} + {\sum }_{j \neq i} {f}_{i j} ( 1 - {w}_{i j} ) & j = i\end{matrix} . \qquad ( 3 )$$

Using this prescription, it is easy to see that the detailed balance condition given by:

$${W}_{i j} {P}_{i}^{e q} = {W}_{j i} {P}_{j}^{e q} \qquad ( 4 )$$

is fulfilled. In many practical implementations the proposal probability is often chosen to be independent of the configuration, i.e., fᵢⱼ=fⱼᵢ. If the proposals are not symmetric, this acceptance is often referred to as Metropolis-Hastings criterion. There are many ways to propose configurational changes to the system, which then constitute the move set. The “optimal” set depends highly on the system and its parameters. Typically, for polymetric systems, local updates of atom positions are complemented by large conformational changes such as pivot moves at higher temperatures, and bond-rebriding moves at low temperature. Due to above prescription, the subsequent states will be chosen from the target equilibrium distribution. Of course, there is some initial relaxation period (also called burn-in time), in which the system is not yet equilibrated, i.e., the configurations are not yet picked from the target distribution. Indeed, while often discarded, also this initial period is of interest, allowing for the study of many nonequilibrium processes, which even allow the extraction of equilibrium exponents in some cases.

While in Monte Carlo of a classical system the only concern is the (potential) energy of the system given by the particles positions x, in molecular dynamics simulations the combined distribution of coordinates and velocities q, i.e., ξ=(x, q). Further, in standard molecular dynamics, the simulation in microcanonical ensemble, manifesting in conserved total energy H(ξ)=H(x, v)=T(v)+U(x), where the potential energy U depends only on the coordinates x and the kinetic energy T(v) depending on the momenta v. This is achieved by iteratively integrating the equation of motions, where the most common prescription used is the velocity-verlet algorithm, consisting of the following steps:

$$v ( t + \frac{1}{2} Δ t ) = v ( t ) + \frac{1}{2} a ( t ) Δ t \qquad ( 5 a )$$

$$x ( t + Δ t ) = x ( t ) + v ( t + \frac{1}{2} Δ t ) Δ t \qquad ( 5 b )$$

$$Recalculate a ( t + Δ t ) from the new positions \qquad ( 5 c )$$

$$v ( t + Δ t ) = v ( t + \frac{1}{2} Δ t ) + \frac{1}{2} a ( t + Δ t ) Δ t \qquad ( 5 d )$$

This integrating step is performed iteratively, until enough statistics is gathered to estimate the desired properties.

In order to sample H(x, v) in the canonical ensemble, a way to control the velocities should be found, for which there is no natural way. That means, there are many ways to achieve this, which have certain advantages and disadvantages. In the canonical ensemble for molecular dynamics, one reproduces the Boltzmann distribution of the total energy:

$${P}^{e q} ( ξ ) = {P}^{e q} ( x , v ) = \frac{{e}^{- H ( x , v ) / k T}}{\int {e}^{- U ( x ) / k T} d x \int {e}^{- T ( v ) / k T} d v} , \qquad ( 6 )$$

which factorizes into the canonical distribution of the potential energy and of the momenta:

$${P}^{e q} ( x , v ) = \frac{{e}^{- U ( x ) / k T}}{\int {e}^{- U ( x ) / k T} d x} \frac{{e}^{- T ( q ) / k T}}{\int {e}^{- T ( v ) / k T} d v} = {P}^{e q} ( x ) {P}^{e q} ( v ) . \qquad ( 7 )$$

Not all thermostats produce the canonical ensemble, so special care has to be taken to make the right choice. Especially, some thermostats are only canonical in P^{eq}(x), but not in the joint distribution P^{eq}(x, v).

Hamiltonian Monte Carlo combines elements from molecular dynamics and Monte Carlo: Micro-canonical molecular dynamics simulations are used as proposals for the Monte Carlo accept/reject step. Here, again both particle positions and velocities are available as the state, i.e., ξ=(x, v). The procedure is as follows:

Draw velocities qᵢ according to Maxwell-Boltzmann distribution, generating the initial state ξᵢ=(nᵢ, qᵢ). This sets a new level of total energy H(ξᵢ).

Propagate the system according to Eqs. (5) for L times with fixed Δt, resulting in the proposal configuration ξⱼ. The integration steps are performed in the microcanonical ensemble, corresponding to conserved total energy H (apart from numerical integration errors).

The new state ξⱼ is then accepted according to Eq. (2). If the proposal is rejected, the system is reset to x=nᵢ and one continues at step 1.

Since the microcanonical integration form ξᵢ to ξⱼ is deterministic, invertible, and volume-preserving one has fᵢⱼ=fji. In principle, to make the whole update reversible, one would need to invert the velocities q before performing step 3, but in practice the kinetic energy is invariant to inversion and new velocities are drawn anyway in step 1. This prescription produces a canonical distribution of the total energy, which trivially following Eq. (5) produces the target distribution of Eq. (1). This replaces (or actually can augment) the hand-crafted move sets used in a standard Monte Carlo simulation. The advantage of this approach is the additional use of the gradient information, which points in the direction of lower energy conformations, drastically improving the acceptance probability while maintaining relatively large conformational changes. In fact, apart from numerical error introduced in the integration, these proposed updates have an acceptance probability close to 100%, since the total energy is conserved.

In recent years there have been approaches to generalize Hamiltonian Monte Carlo to non-volume preserving integration prescription, i.e., to change the propagator of the previous sections in such a way that f(ξⱼ, ξᵢ)≠f(ξᵢ, ξⱼ) in Eq. (2). For an arbitrary update of ξᵢ given by the functional expression g(ξᵢ)=ξⱼ, the determinant of the Jacobian matrix acts as scaling factor between the phase-space volumes (see the standard formula for the change of variables used, e.g., in integration), i.e., Eq. (2) reads:

$${w}_{i j} = min ( 1 , \frac{{P}^{e q} ( {ξ}_{j} )}{{P}^{e q} ( {ξ}_{i} )} \vert det ( \frac{\partial g ( {ξ}_{i} )}{\partial {ξ}_{i}} ) \vert ) \qquad ( 8 )$$

For an efficient update procedure, there is a need to be able to calculate the determinant of the Jacobian matrix J=∂g(ξᵢ)/∂ξᵢ fast. In previous references, it was proposed to limit the form of the function g, i.e., to ensure that the update only changes one of the variables at a time and that no sub-update depends non-linearly on any of the variables being updated. Then, the Jacobian J is a triangular matrix and the determinant is simply given by the trace |Tr(J)|=|det(J)|.

In Levy, it was proposed to alter the velocity-verlet update of Eq. 5 to include rescaling functions which depend only on variables not being updated. An embodiment of the present invention follows the general idea of this approach, resulting in the following update prescription:

$${v}_{i}^{′} = {v}_{i} {D}_{i}^{v} ( \{ {x}_{j} \} , \{ {a}_{j} \} ) + \frac{1}{2} {a}_{i} {D}_{i}^{a} ( \{ {x}_{j} \} , \{ {a}_{j} \} ) Δ t \qquad ( 9 a )$$

$${x}_{i}^{′} = {x}_{i} + {v}_{i}^{′} Δ t \qquad ( 9 b )$$

Calculate

$${a}_{i}^{′}$$

from the new positions:

$$\{ {x}_{j}^{′} \} \qquad ( 9 c )$$

$${v}_{i}^{″} = {v}_{i}^{′} {D}^{v} ( \{ {x}_{j}^{′} \} , \{ {a}_{j}^{′} \} ) + \frac{1}{2} {a}_{i}^{′} {D}^{a} ( \{ {x}_{j}^{′} \} , \{ {a}_{j}^{′} \} ) Δ t \qquad ( 9 d )$$

where the diagonal matrices D^{v} and D^{a} have been introduced in Eq. (9a) and (9d). The matrices are currently of diagonal form:

$${D}_{i}^{v , a} ( \{ {x}_{j} \} , \{ {a}_{j} \} ) = ( \begin{matrix}{D}_{i , 0}^{v , a} ( \{ {x}_{j} \} , \{ {a}_{j} \} ) & 0 & 0 \\ 0 & {D}_{i , 1}^{v , a} ( \{ {x}_{j} \} , \{ {a}_{j} \} ) & 0 \\ 0 & 0 & {D}_{i , 2}^{v , a} ( \{ {x}_{j} \} , \{ {a}_{j} \} )\end{matrix} ) \qquad ( 10 )$$

and act as independently scaling each component of v or a, respectively. Here,

$${D}_{k}^{v , a} ( x ( t ) , a ) ( t )$$

are a priori, arbitrary functions, which depend only on x, a, and potentially other properties of the system, that, however, do not depend on v. To calculate the (determinant) of the Jacobian J associated with this sequence of updates in Eqs. 9(a, b, d), each equation has to be read as keeping the other variables of the transformation unaltered. One can further show that the total determinant of the Jacobian of a transformation is given by just the multiplication of the individual determinants of each individual transformation. The total determinant of this combined transformation g is then:

$$\vert det ( J ) \vert = \vert det ( {D}^{v} ( \{ {x}_{j} \} , \{ {a}_{j} \} ) \vert \cdot \vert det ( {D}^{v} ( \{ {x}_{j}^{′} \} , \{ {a}_{j}^{′} \} ) ) \vert \qquad ( 11 )$$

Unlike the velocity-verlet update described in Eqs. (5)(a-d) used in Hamiltonian Monte Carlo, the update is not made reversible by picking a random velocity at the beginning, so that here one also has to implement the reverse direction of the update. For this, every equation itself is inverted, and the time steps are performed from the latest time to the earliest time.

As mentioned in the last section, arbitrary functions D^{v,a}(x(t), a(t)) may be chosen in the update. This, of course, means a parameterized expression can be found, which may be used to improve the sampling efficiency. However, it is challenging to canonically find an expression to achieve this. Luckily, this is where machine-learning thrives, i.e., the need to (explicitly) parameterize the function D^{v,a}(x(t), a(t)) is replaced and instead high-dimensional functions with many parameters are relied on. The values of those parameters are optimized towards the underlying task by minimizing the defined loss function; this is referred to as unsupervised learning. Thus the basic principle of machine learning is used to leverage data to “learn” underlying patterns in order to improve performance on some set of tasks.

One of the most common neural network architecture are feed-forward neural networks (often also called multi-layer perceptron). In those, the input is just a (real-valued) vector x₀∈ [symbol] of size n. Then a couple of so-called hidden layer follow. In each hidden layer, the input data is multiplied by a matrix and a bias term is added. For the first layer, one has a matrix m₀∈ [symbol] and a bias term b₀∈ [symbol] In fact, however, this would be a purely linear relationship, so additionally one introduces a (non-linear) activation function σ, which acts on each element of the output vector. The resulting values of the first hidden unit x₁ are then given by:

$${x}_{1} = σ ( {m}_{0} {x}_{0} + {b}_{0} ) . \qquad ( 12 )$$

Of course, the output depends on the values of m₀ and b₀; in fact, the values (or often called weights) is what is “learned” in a neural network. These layers are repeated many times to form a deep neural network, i.e., one has for the layer number l+1:

$${x}_{l + 1} = σ ( {m}_{l} {x}_{l} + {b}_{l} ) , \qquad ( 13 )$$

which depends only on the layer 1 explicitly (akin to a Markov chain). Here, every matrix mi has as input dimension the size of the previous layer and subsequently the chosen output dimension specifies the input into the next layer. The final layer output is used to calculate the (application) defined loss L, characterizing the deviation from the target. A typical choice for the activation function is the “rectified linear unit” defined as:

$$σ ( x ) = \{ \begin{matrix}x , & x > 0 \\ 0 , & x \leq 0\end{matrix} \qquad ( 14 )$$

The parameters of this neural network are then updated depending on the loss using back propagation, i.e., the derivative of the loss with respect to every parameter mi is calculated and then used to calculate the change of the parameter:

$${m}_{i}^{′} = {m}_{i} - η \frac{\partial L}{\partial {m}_{i}} \qquad ( 15 )$$

where η is the so-called learning rate and mi is an arbitrary parameter of the network (e.g., one value of the matrix mₗ or a single bias value bₗ). This type of update rule is also called gradient descent. Variants of this exist, which for example also include a momentum variable to accelerate convergence. One of the most popular optimizers incorporating such additional terms, i.e., the Adam optimizer (see, e.g., Kingma, D. Ba, J. L., “ADAM: A Method for Stochastic Optimization,” arXiv:1412.6980 (January 2017), which is hereby incorporated by reference herein) is used.

As in any machine learning setting, a loss is defined which should be optimized by the optimizer. One of the most natural definition of the loss in this set-up is:

$$L = - {{p}_{x} ( {x}^{′} - x )}^{2} \qquad ( 16 )$$

where pₓ is the acceptance probability according given by the Metropolis-Hastings criterion of Eq. (2) and (x′−x)² is the movement in coordinate phase-space. For molecular systems this definition, however, is potentially insufficient: Translations lead to a major decrease in L, whereas in reality the physics of the system has not changed. To circumvent this, two adaptations are presented: i) Calculating the matrix consisting of relative distances and take these as loss term or ii) Learning parts of the whole matrix presented below.

The ultimate performance of the learned parameters is evaluated by investigating the autocorrelation times of the potential energy, defined via the autocorrelation function

$$A ( k ) = \frac{〈 {𝒪}_{i} {𝒪}_{i + k} 〉 - 〈 {𝒪}_{i} 〉 〈 {𝒪}_{i} 〉}{〈 {𝒪}_{i}^{2} 〉 - 〈 {𝒪}_{i} 〉 〈 {𝒪}_{i} 〉} \qquad ( 17 )$$

which gives the integrated autocorrelation time

$${τ}_{𝒪 , int} = \frac{1}{2} + {\sum }_{k = 1}^{N} A ( k ) ( 1 - \frac{k}{N} ) \qquad ( 18 )$$

This has the interpretation of (local) exploration speed and needs to be accounted for when calculating the standard deviation on observables as

$${σ}_{\overline{𝒪}}^{2} = \frac{{σ}_{𝒪}^{2}}{N} 2 {τ}_{𝒪 , int} \qquad ( 19 )$$

Indeed, it can be shown that the above loss in Eq. (16) reduces the autocorrelation time at k=1 [ ].

The approach is presented for the common test system: Alanine-Dipeptide, which is often used to test novel simulation methods. As force-field Amber 19ffSB is used and simulated in vacuum. The goal is to simulate in the canonical ensemble at 300K. The results are compare with MD simulations obtained using a Langevin thermostat with drag. For the Hamiltonian Monte Carlo simulations, the number of MD steps are fixed (calls to the integrator) to 20. The backward pass updating the model parameters is performed every 10 Hamiltonian Monte Carlo steps, which has the interpretation of epoch in the machine learning language. In all experiments, the system is initially equilibrated for 2000 epochs (burn-in period), followed by a period in which the parameters of the model (“training”) and finally the evaluation of the performance of the method (“test”) are learned. As optimizer, the Adam is used with default parameters, using different learning rates.

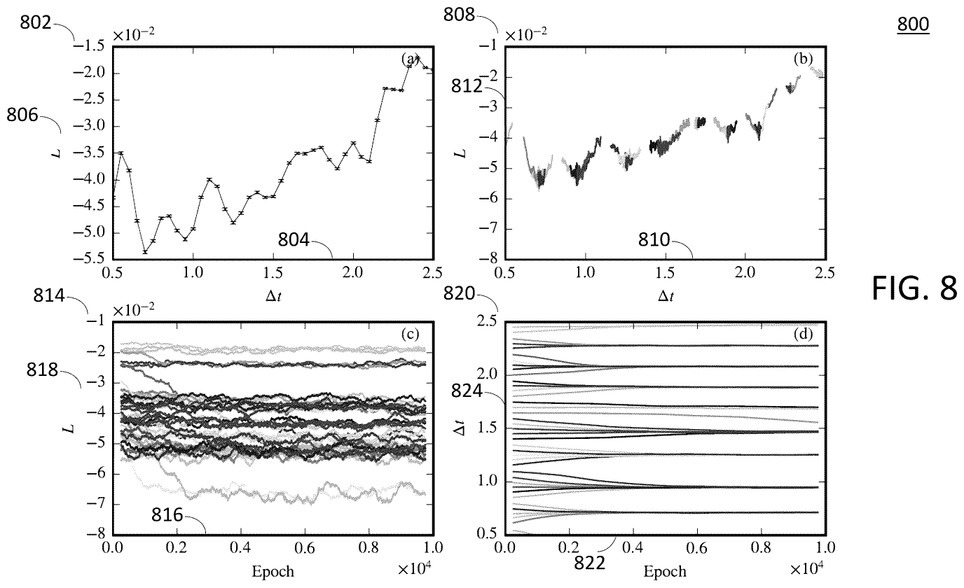

As a first step, the learning of the ideal time step Δt in Eqs. (9) is discussed. In FIG. 8 the values of the loss defined in Eq. 16 for different values of Δt are shown. FIG. 8 graphically shows experimental results of Loss L of Alanine-Dipeptide in dependence of the time step Δt. FIG. 8 includes graphs 802, 808, 814, and 820. Graphs 802 and 808 plot Loss L on the respective y-axes 806 and 812 and Δt on the respective x-axes 804 and 810. Graph 814 plot Loss L on the y-axis 818 and Epoch on the x-axis 816. Graph 820 plots Δt on the y-axis 824 and Epoch on the x-axis 822.

Graph 802 shows the data obtained by fixing the time step without learning. The loss landscape clearly has several minima separated by barriers. The error bars are calculated taking the autocorrelation into account. In graphs 808, 814, and 820 the same initial set-up as graph 802 is used, but the value of the time step can be learned. In graph 808, the plot is equivalent to the plot as in graph 808, showing that depending on the starting value of the value is optimized towards the closes local minimum. In graph 814 the loss as a function of training epoch is shown and in graph 820, the corresponding value of Δt during training is shown.

The signature of a rugged loss landscape is seen, illustrated by several minima which are separated by barriers. If attempts are made to learn the optimal value of Δt, the next local minima is approached (with local gradient based optimizers). Smaller barriers may be overcome by the adaptive optimizer used (Adam).

It is interesting to extend this study to larger systems in general and systems with explicit solvent in general.

In some embodiments, the study can be combined with hand-crafted updates, which can be used in conjunction with the learned integration to propose changes to the system. For example the pivot-move has been proven to be exceptionally efficient in the high-temperature phase to decorrelate samples.

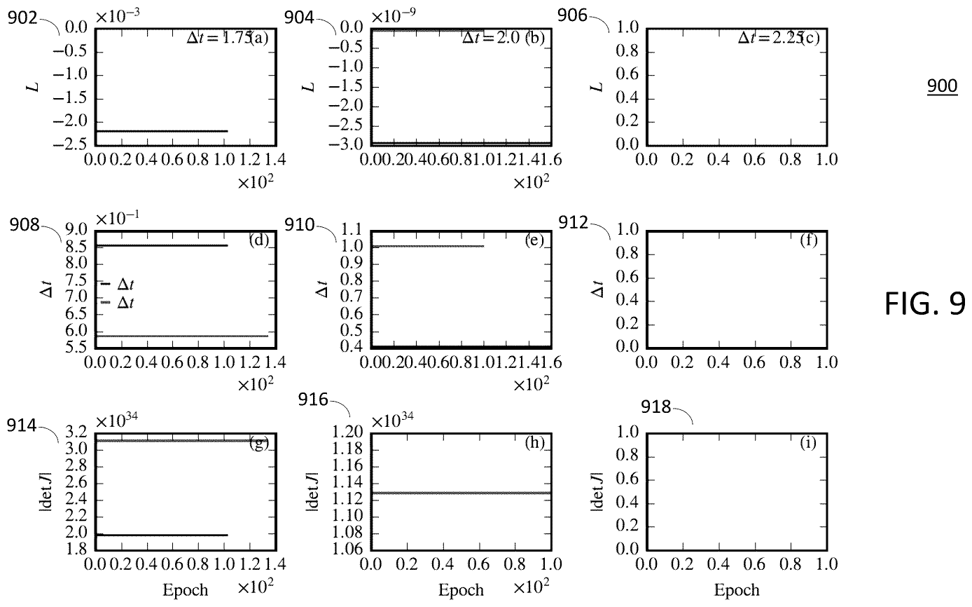

FIG. 9 graphically shows experimental results of Loss L as a function of training epochs for an initial value of Δt of (a) 1.0, (b) 1.5, and (c) 2.0, in accordance with embodiments of the present invention. FIG. 9 includes graphs 902-918. Graphs 902, 904, and 906 plot Loss L on the y-axis and Epoch on the x-axis. Graphs 908, 910, and 912 plot Δt on the y-axis and Epoch on the x-axis. Graphs 914, 916, and 918 plot |det J| on the y-axis and Epoch on the x-axis.

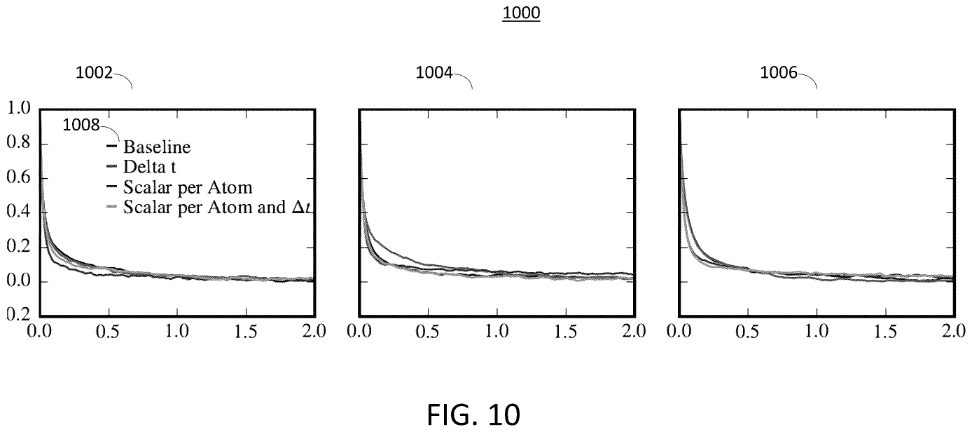

FIG. 10 graphically shows autocorrelation results, in accordance with embodiments of the present invention. FIG. 10 includes graphs 1002, 1004, and 1006.

In the direction of machine-learning, the development of new network architectures is a very active field of research, until now mostly focused on the development of force-fields. In some embodiments, advances in this field are applied to the problem to see improvements in accuracy and speed. Finally, of course the exact form of the integration steps and where to incorporate the effect of neural networks is not fixed. For example, contrary to the usual trade-off observed in classical molecular dynamics, it might prove beneficial to use higher-order integrators.

The following provides additional further background and description of exemplary embodiments of the present invention, which may overlap to some extent with some of the information provided above. To the extent the terminology used to describe the exemplary embodiments may differ from the terminology used to describe the above embodiments, a person having skill in the art would understand that certain terms correspond to one another in the different embodiments. Features described in the following can be combined with features described above in various embodiments.

The performance of Hamiltonian Monte Carlo crucially depends on its parameters, in particular the integration time step and the number of integration steps. An adaptive general-purpose framework is presented to automatically tune these parameters based on a loss function which promotes the fast exploration of phase space. For this, a fully-differentiable set-up is used along with backpropagation for optimization. An attention-like loss is defined which allows for the gradient driven learning of the distribution of integration steps. The importance of jittering for a smooth loss-surface is highlighted. The approach is demonstrated for the one-dimensional harmonic oscillator and alanine dipeptide, a small protein common as a test-case for simulation methods. A good correspondence between the loss and the autocorrelation times is found, resulting in well-tuned parameters for Hamiltonian Monte Carlo.

Simulations of biomolecular systems are predominately performed using either molecular dynamics (MD) or (random walk) Monte Carlo (MC) simulations. While the former method, in its basic formulation, solves Newton's equations numerically, the latter (directly) samples from the target distribution. Both approaches can be used to sample the canonical ensemble, i.e., a system in contact with a thermal bath and can, for many purposes, be used as plug-in replacements for sampling (as long as dynamic properties are not needed). A priori, there is thus no apparent advantage of one method over the other.

In molecular dynamics, time needs to be discretized, and the equation of motions are integrated numerically. This introduces short-time errors as well as accumulating errors, leading for example to a violation of energy conversation (for microcanonical simulations) in the long-time limit. While small enough time steps are important for the correct physical evolution (small deviations of the simulated Hamiltonian), they lead to severely impeded dynamics since the system only slowly moves through phase-space. Additional complications are introduced when the system is coupled to a thermostat; there, the velocities as proxy for the system's temperature are modified. There is no canonical way to do this, manifested by many alternative approaches available in the literature.

In MC, on the other hand, there is no error introduced by numerical integration and no adverse effects from controlling the temperature, as this is directly manifested in the acceptance probability. However, the generate trial configurations need to be generated in a clever way, i.e., to come up with a move set which constitutes major configuration changes without being penalized by a very low acceptance. There are a couple of well accepted move-sets for different systems incorporating prior knowledge which are based on intuition/heuristic arguments. However, by definition, finding an optimal move set is not possible. Traditionally, the proposed moves are random, i.e., their proposal probability does not depend on the current configuration in order to guarantee detailed balance.

In the literature, there already exist a couple of approaches to combine both methods, where one of the most studied one is Hamiltonian Monte Carlo (HMC), which originally was introduced as hybrid Monte Carlo. The basic idea is to propagate the system using a (microcanonical) integrator for a given number of steps which conserves the total energy as used in MD simulations, but add an acceptance of proposals obtained in this way using MC. In case of rejection of a move, the system is reset (in the typical MC fashion) to the previous state. The additional step required for HMC is to randomly draw new velocities, as otherwise the resulting configuration would be identical after rejection. Compared to the individual methods on their own, this combined approach has the advantage that there is no adverse effect of the numerical integration or external control of temperature, while providing a systematic way to generate trial configurations.

The trial configurations generated by this approach can have a high acceptance probability, especially for small time steps and few integration steps, as then both the numeric error and the integration error is small (the total energy is in principle conserved by the integrator). Choosing both these parameters small, however, leads again to slow phase space exploration, whereas choosing them large results in the simulation of a shadow Hamiltonian and fast accumulation of numeric errors resulting in small acceptance rates. This implies that there is some balance between those two effects, for which the simulation is much faster at exploring the phase-space.

A fully-differentiable framework will be presented which allows to tune both these parameters via backpropagation based on a local loss definition.

Further, the integrator is extended to include atom-dependent time steps, which would be very difficult to optimize for using heuristic (gradient uninformed) methods. Two systems are investigated: The one dimensional harmonic oscillator where the focus is on the fundamental mechanisms of the method before turning to demonstrate its performance for the common test system alanine dipeptide. A short overview is given over the involved simulation methods, followed by a conceptual introduction to the employed fully-differentiable set-up and loss definition. Results for both systems are presented, discussing the achieved improvements in simulation speed and an analysis of the learning process and obtained parameters. Finally, conclusions and outlooks about future research is given.

In the following, the goal is to simulate a classical system, which consists of particles/atoms interacting via classical potentials. This system is in coupled to a heatbath, i.e., the target is to simulate in the canonical ensemble. Then, each microstate described by the coordinates x occurs with a probability that is given by:

$${P}^{e q} ( x ) = \frac{1}{Z} {e}^{- \frac{U ( x )}{k T}} , \qquad ( 1 )$$

where Z=∫e^{-U(x)/kT} dx is the partition function (in statistic often simple referred to as normalizing constant), U(x) is the (potential) energy of a microstate depending on the atoms positions, k is the Boltzmann constant, and T is the temperature of the heatbath. While the evaluation of the partition function would provide access to many thermodynamic observables, this is in practice not possible since this necessitates a summation over all possible microstates. Instead, attempts are made to approximate expectation values of quantities of interest at a fixed temperature by producing samples from the target distribution, utilizing methods that do not rely on the value of Z.

In MC, producing samples from the target distribution is conceptually, a priori, straightforward. The main idea behind a Monte Carlo simulation is to build a Markov chain, starting with a random configuration of the system at interest and subsequently progressing by proposing new configurations (which only depend on the current configuration). There is some freedom in choosing the transition probabilities Wᵢⱼ=W(ξᵢ, ξⱼ) between microstate ξᵢ, ξⱼ. In MC for molecular systems, typically ξ=x, i.e., a set of Cartesian coordinates, but in general this can be any configurational information. One of the most flexible choices for the acceptance criterion between states is the original Metropolis algorithm which reads:

$${w}_{ij} = w ( {ξ}_{i} , {ξ}_{j} ) = min ( 1 \frac{f ( {ξ}_{j} , {ξ}_{i} )}{f ( {ξ}_{i} , {ξ}_{j} )} \frac{{P}^{eq} ( {ξ}_{j} )}{{P}^{eq} ( {ξ}_{i} )} ) , \qquad ( 2 )$$

Where fᵢⱼ=f(ξᵢ, ξⱼ) is the proposal probability for a potential update to a new microstate. Here, the partition function cancels, since one is only interested in the ratio of the equilibrium distributions. This then leads to the transition probability:

$${W}_{ij} = \{ \begin{matrix}{f}_{ij} {w}_{ij} & j \neq i \\ {f}_{ii} + {\sum }_{j \neq i} {f}_{ij} ( w - {w}_{ij} ) & j = i\end{matrix} \qquad ( 3 )$$

Using this prescription, it is easy to see that the detailed balance condition given by:

$${W}_{i j} {P}_{i}^{e q} = {W}_{j i} {P}_{j}^{e q} \qquad ( 4 )$$

is fulfilled. This is a sufficient condition for the convergence to the equilibrium distribution. In many practical implementations the proposal probability is often chose to be independent of the configuration, i.e., fᵢⱼ=fⱼᵢ. If the proposals are not symmetric, this acceptance is often referred to as Metropolis-Hastings criterion. There are many ways to generate configurational changes to the system, which then constitute the move set. The “optimal” set depends highly on the system and its parameters. Typically, for polymeric systems, local updates of atom positions are complemented by large conformational changes such as pivot moves at higher temperatures, and bond-rebriding moves at low temperature. States first represent the equilibrium distribution after an initial relaxation/burn-in period. Indeed, while often discarded, this initial period is also of great interest, allowing for the study of many nonequilibrium processes, which even facilitate the extraction of equilibrium exponents in some cases.

While in MC of a classical system one is only concerned with the (potential) energy of the system given by the particles positions x, in MD one simulates the combined distribution of coordinates and velocities v, i.e., one has ξ=(x, v). Further, in standard MD, one simulates in the microcanonical ensemble, manifesting in conserved total energy H(ξ)=H(x, v)=U(x)+K(v), where the potential energy U(x) depends only on the coordinates x and the kinetic energy K(v) depending on the momenta v. This is achieved by iteratively integrating the equation of motions, where the most common prescription used is the velocity Verlet algorithm, consisting of the following steps:

$${v}_{i} ( t + \frac{1}{2} Δ {t}_{i} ) = {v}_{i} ( t ) + \frac{1}{2} {a}_{i} ( t ) Δ {t}_{i} \qquad ( 5 a )$$

$${x}_{i} ( t + Δ {t}_{i} ) = {x}_{i} ( t ) + {v}_{i} ( t + \frac{1}{2} Δ {t}_{i} ) Δ {t}_{i} \qquad ( 5 b )$$

Recalculate aᵢ(t+Δtᵢ) from the new positions (5c)

$${v}_{i} ( t + Δ {t}_{i} ) = {v}_{i} ( t + \frac{1}{2} Δ {t}_{i} ) + \frac{1}{2} {a}_{i} ( t + Δ {t}_{i} ) Δ {t}_{i} \qquad ( 5 d )$$

where i is the index of the atom, Δtᵢ is the (atom dependent) timestep, and aᵢ=−m⁻¹∂/∂xᵢU(x) is the acceleration acting on the atom obtained from the potential. This integrating step is performed iteratively, until enough statistics is gathered to estimate the desired properties. In a standard MD simulation, the individual atoms need to evolve synchronously in time, which practically restricts the use of time step to a global definition of Δtᵢ=Δt, i.e., the timestep is not dependent on the atom index. That also implies that the maximal timestep which can be used is determined by the fastest mode of oscillation.

To sample from P^{eq} in the canonical ensemble using MD, a method to control the velocities can be found, for which there is no natural way. That means, there are many ways to achieve this, which have certain advantages and disadvantages. In the canonical ensemble for MD, the Boltzmann distribution of the total energy is reproduced:

$${P}^{e q} ( ξ ) = {P}^{e q} ( x , v ) = \frac{{e}^{- H ( x , v ) / kT}}{\int {e}^{- U ( x ) / kT} d x \int {e}^{- K ( v ) / kT} dv} , \qquad ( 6 )$$

which factorizes into the canonical distribution of the potential energy and of the momenta

$${P}^{e q} ( x , v ) = \frac{{e}^{- U ( x ) / kT}}{\int {e}^{- U ( x ) / kT} dx} \frac{{e}^{- K ( v ) / kT}}{\int {e}^{- K ( v ) / kT} d v} = {P}^{e q} ( x ) {P}^{e q} ( v ) . \qquad ( 7 )$$

Not all thermostats produce the canonical ensemble, so special care has to be taken to make the right choice. Especially, some thermostats are only canonical in P^{eq}(x), but not in the joint distribution P^{eq}(x, v).

In some embodiments, sampling using molecular dynamics is only approximate, i.e., the convergence to the target distribution is only guaranteed in the limit of Δt→0. In contrast, MC sampling is asymptotically exact.

HMC combines elements from molecular dynamics and MC: Microcanonical MD simulations are used as proposals for the MC accept/reject step. This combination of method was originally generated by Duane et al. and later popularized in the statistics community with applications towards inference of Bayesian neural networks. While recent work exists highlighting the performance of HMC, and it is implemented in or for commonly used simulation packages, it is often not the go-to method of choice. In part, this is probably due to the belief that this method becomes ineffective for simulations of large and complex systems, although recent work suggests otherwise. Another part lacking in its acceptance is the problem of choosing optimal parameters of the simulation, as will become clear in the following.

For this method, both particle positions and velocities are present as the state, i.e., ξ=(x, v). While the interpretation of having a velocity is quite natural from a physical point of view, it is not as straight forward in many other fields of applications, for which one talks about extending the phase-space. Indeed, also in the physics literature one finds instances where it is not clear how to define the velocities. The steps of HMC are as follows:

Draw velocities vᵢ according to Maxwell-Boltzmann distribution, generating the initial state ξᵢ=(xᵢ, vᵢ). This sets a new level of total energy H(ξᵢ).

Propagate the system according to Eqs. (5) for n steps with fixed Δtᵢ, resulting in the proposal configuration ξⱼ. The integration steps are performed in the microcanonical ensemble, corresponding to in principally conserved total energy H (in practice, this is of course not the case due to the discretization in time).

The new state (j is then accepted according to Eq. (2). If the proposal is rejected, the system is reset to x=xᵢ and one continues at step 1.

There are some important details of this procedure which will be discussed in the following. In practice, it is beneficial to not use constant Δtᵢ but to rather pick from a distribution. This is referred to as jittering and avoids some problems related to repeatedly running into small unfavorable regions in phase-space due to the deterministic dynamics. In the simulations, the time steps are picked from a normal distribution with fixed relative variance, i.e.:

$${Δt}_{i}^{′} ∼ 𝒩 ( {Δ t}_{i} , {s Δ t}_{i} ) \qquad ( 8 )$$

where s is a free parameter. The influence and associated problems of not jittering the time step are shown later.

Since the microcanonical integration form ξᵢ to ξⱼ is deterministic, invertible, and volume-preserving, the ratio of the target distributions P^{eq} in Eq. 2 can be used since this implies that the proposal probability fᵢⱼ exactly cancel.

In principle, to make the whole update reversible (necessary to fulfill detailed balance), the velocities v can be inverted before performing step 3, but in practice the kinetic energy is invariant to inversion of velocities and new velocities are drawn anyway in step 1. This also eliminates the need to explicitly implement the forward and backward integration otherwise needed for detailed balance, as the velocities are picked randomly either way. The overall prescription produces a canonical distribution of the total energy, which following Eq. (2) produces the target distribution of Eq. (1).

HMC replaces (or actually can augment) the handcrafted move sets used in a standard MC simulation. The advantage of this approach is the additional use of the gradient information, which allows for informed moves that either dissipate or absorb kinetic energy. This way, the acceptance probability is drastically improved while maintaining relatively large conformational changes. In fact, apart from numerical error introduced in the integration, these updates have an acceptance probability close to 100%, since the total energy is conserved.

For an effective exploration of phase space, the phase space movement and acceptance rates can be balanced. Indeed, it has been shown in Beskos that the optimal acceptance probability should approach α=0.651 for (vanilla) HMC (under some assumptions using the standard leapfrog integrator), independent of the particular (high dimensional) target. In practice, however, it is not clear if all assumptions hold and it has been found to sometimes perform poorly, especially due to the observation that samplers with the same acceptance rate can exhibit vastly different behavior.

The practical solution often employed is to prescribe a target acceptance rate of around 50% to 60% and heuristically optimize the time step Δt to take such a value that this is observed on average. A common method to set a good number of integration steps n is called NUTS, which uses a recursive algorithm to build a set of candidate configurations on the fly. This procedure stops once the so-called U-Turn condition is satisfied, which signifies a doubling back of the trajectory. While this approach works well in practice, in order to preserve detailed balance, there is a need to both consider moves in forward and backward direction which recursively build a tree of steps, from which one then samples the proposal.

This can incur a two-fold overhead in performed updates. In Hoffman et. al., an approach to tune HMC is proposed: The target acceptance probability was combined with an optimization objective based on multiple chains which is able to tune the total trajectory length nΔt, i.e., to some extent to tune both crucial parameters of HMC. There, it is shown that such an approach can outperform

NUTS and finds the optimal parameters one otherwise would only find using a grid search.

Another direction of research is a reparametrization of the simulation space on which HMC is performed, as e.g. proposed in Riemannian HMC or via a mapping given by learned functions. While these approaches are promising, at least the former appears to suffer from problems when numerically integrating requiring for example hand-tuning of many parameters. A concern for the latter is that the map needs to adequately capture the geometry of the target distribution, as otherwise one would get slow mixing in the tails. These methods still require the setting of Δt and n (or possibly more parameters if a more parameterized version of the integrator) is chosen, i.e., the approach is still be applicable in this setting.

In some embodiments, the propagation of the system can be performed by any arbitrary function and does not need to follow the structure set in Eqs. (5), i.e., proposals do not need to use the velocity Verlet integrator. One example for this is classical random-walk Monte Carlo, where changes to the system are typically performed by picking them from a (configuration independent) distribution. For this approach, it is also possible to learn parameters based on gradient information.

Writing the proposal as an arbitrary function of ξᵢ instead of using the physically integrator, i.e., using

$${ξ}_{i}^{′} = g ( {ξ}_{i} ) ,$$

the connection to normalizing flows becomes clear. In this setting, the determinant of the Jacobian matrix acts as scaling factor between the phase space volumes (see the standard formula for the change of variables used, e.g., in multidimensional integration), i.e., Eq. (2) reads:

$${w}_{iij} ( 1 , \frac{{P}^{eq} ( {ξ}_{j} )}{{P}^{eq} ( {ξ}_{i} )} \vert det ( \frac{\partial g ( {ξ}_{i} )}{\partial {ξ}_{i}} ) \vert ) \qquad ( 9 )$$

Thus, the general requirement on g(ξᵢ) is that the determinant of the Jacobian of the transformation can be efficiently calculated. In practice, several functions can be stacked together, forming again a sequence of function calls. This is indeed what the velocity Verlet integrator does, although in such a form that the determinant of the Jacobian is one and does not need to be considered. Changing the time step or the number of integration steps does not result in a volume changing transformation.

Normalizing flows share the requirement that calculating the determinant of the Jacobian is fast. They are usually used in a somewhat different context and focus on proposing global updates to accelerate sampling, where they are trained to minimize some variant of the Kullback-Leibler divergence for a given target distribution.

In some embodiments, good average parameters of the velocity Verlet integrator presented in Eqs. (5) are found, in particular the time steps Δtᵢ and the number of integration steps n. Since the number of atoms can grow quite large, this constitutes many correlated parameters that cannot efficiently be optimized for using heuristic methods. This makes the use of a fully differentiable framework very appealing, since then gradient information can be used to update all parameters. Although these optimizers, by definition, only use local information and can be trapped in a local minimum, experience has shown that they are still able to very efficiently find suitable values for the parameters, especially in the context of neural networks.

A fully differentiable simulation set-up is achieved by implementing the algorithm in pyTorch, a software library often used in machine learning. The appeal of this approach is automatic differentiation which allows for the evaluation of partial derivatives of a function specified by a computer program. In pyTorch, any operation applied to the so-called tensors is recorded, so that via the chain rule one is able to calculate the gradient on any parameter of the computation graph. To make changes based on this gradient information, a way is then used to judge the goodness of the output of the computation, i.e., a loss L associated with the integration.

The parameters are then updated depending on the loss using backpropagation, i.e., the derivative of the loss with respect to every parameter θᵢ of the computation graph to be tuned is calculated and then used to calculate the change of the parameter:

$${θ}_{i}^{′} = {θ}_{i} - η \frac{\partial L}{\partial {θ}_{i}} \qquad ( 10 )$$

where η is the so-called learning rate and θᵢ is for example the timesteps Δtᵢ or the number of integrations steps n. This type of update rule is also called gradient descent, i.e., the parameters are changed in opposite direction of the gradient. Variants of this optimization algorithm exist, which for example also include a momentum variable to accelerate convergence. In some embodiments, one of the most popular optimizers incorporating such additional terms, i.e., the Adam optimizer will be used.

The most common way to evaluate the performance of an MC simulation is to investigate the autocorrelation between subsequent states of the chain. For this, the definition of the autocorrelation function is recalled:

$${A}_{𝒪} ( k ) = \frac{〈 {\text{?}}_{i} {\text{?}}_{i + k} 〉 - 〈 {\text{?}}_{i} 〉 〈 {\text{?}}_{i} 〉}{〈 {\text{?}}_{i}^{2} 〉 - 〈 {\text{?}}_{i} 〉 〈 {\text{?}}_{i} 〉} , \qquad ( 11 )$$

where [symbol] . . . [symbol] symbolizes the thermodynamic expectation value in equilibrium (after equilibration) when sampling P^{eq}. From the autocorrelation function, one way to obtain the autocorrelation time is:

$${τ}_{𝒪} = \frac{1}{2} + {\sum }_{k = 1}^{N} A ( k ) ( 1 - \frac{k}{N} ) \qquad ( 12 )$$

Here, [symbol] is any observable of the system, for example the potential energy which will be used in the following this implies a different autocorrelation time per observable). The autocorrelation time is related to the effective sample size (ESS):

$${N}_{𝒪 , eff} = \frac{N}{{2}_{{τ}_{𝒪}}} \qquad ( 13 )$$

In practice, the ESS is calculated as implemented in tensor flow where the sum in Eq. (12) is truncated as proposed in Geyer. The ESS is then used to estimate the autocorrelation time [symbol] via above relation.

The importance of the autocorrelation time lies in the need to be included when calculating the standard deviation on observables as:

$$σ \frac{2}{𝒪} = \frac{{σ}_{𝒪}^{2}}{{N}_{𝒪 , eff}} = \frac{{σ}_{𝒪}^{2}}{N} 2 {τ}_{𝒪} . \qquad ( 14 )$$

That is, when an algorithm has a smaller autocorrelation time one needs to simulate shorter to achieve the same error on the observable.

However, quantities related to the autocorrelation function cannot be effectively used as an objective for the fully-differentiable set-up since they require long chains to provide reliable estimated of [symbol] . Therefore, a proxy loss is used to the autocorrelation time, defined as:

$${L}_{n} = - {p}_{n} {\vert {x}_{n}^{′} - {x}_{0} \vert}^{b} \qquad ( 15 )$$

where pₙ is the acceptance probability of the proposal and

$${\vert {x}_{n}^{′} - {x}_{0} \vert}^{b}$$

is the movement in coordinate phase-space (distance between the start and end states), computed after performing n integration steps, i.e., for each proposal generated. A common choice is b=2 where one is thus optimizing for the expected squared jump-distance. This definition, however, is not unique and not guaranteed to provide the best correspondence to a reduction in autocorrelation times. For example, an optimal jump in real coordinates does not need to lead to an optimal autocorrelation time for other observables such as the potential energy. Further, the expected jump distance only optimizes for the lag-1 autocorrelation, whereas further values are ignored. It is not clear how well the information about correlations at small lags correlates with the overall shape of the autocorrelation function. This means that there is still some freedom in optimizing the loss function. For example in Levy the authors introduced an additional reciprocal term which penalized small jumps more strongly and in Refs. 33 and 55 alternative definition relying on multiple chains are proposed, either by evaluating the change in the estimators of the expected squared jump or focusing on difficult directions. As will be shown, simply setting b>2 provides a better correspondence with the autocorrelation times observed for the potential energy.

Since the goal is to propagate the system as efficiently as possible in terms of computational effort, the definition of the loss in Eq. (15) is only sufficient when the number of integration steps n is fixed. In some embodiments, a rescaling of the loss is introduced by the computational effort, i.e., the loss is defined as Lₙ/n and by this incorporate the information that every integration step takes roughly the same computational effort. Previously, it was empirically found that defining L/\sqrt{n} provides a well working approach in practice, although it is not clear why the cost should not enter linearly.

When trying to optimize for n a way to include the number of steps n into the computation graph can be found which allows calculating partial derivatives with respect to it and gives a signal for good values. Thus, to learn the optimal (distribution of the) number of integration steps n, weighting the output of every integration step with a learned distribution is performed. For this, the loss is defined as:

$$L = {\sum }_{n = 1}^{N} {c}_{n} {L}_{n} / n , \qquad ( 16 )$$

where cₙ are the weights of the particular number of integration steps and N is the maximal number of integration steps considered during training. The cₙ are in practice obtained as softmax of unrestricted parameters Cₙ, i.e.,

$${c}_{n} = σ ( {C}_{n} ) = \frac{{e}^{{C}_{n}}}{{\sum }_{n = 1}^{N} {e}^{{C}_{n}}} , \qquad ( 17 )$$

where the temperature of the softmax is set to unity. Cₙ is initialized as uniform random number from zero to one and apply the softmax to arrive at the initial cₙ. This approach is inspired by an attention-like set-up and allows to give “attention” towards a particular integration step. While this approach makes it necessary to set a maximum number of integration steps N during training and always simulate until the maximum is reached, after learning one categorically picks from the probabilities and there is thus no unnecessary computing. While the approach provides a fixed distribution over the steps n, e.g., NUTS gives a good number of steps which depend on the particular trajectory. However, some integrations steps can be wasted since in order to preserve detailed balance both forward and backward steps need to be executed, so this trade off may be beneficial nonetheless, as already shown in M. Hoffman. The picking from cₙ can also serve as a jittering on the number of integration steps and thus helps to avoid problems like local minima in the loss-surface, see the discussion on jittering for Δt.

In practice, the loss by the number of atoms (usually fixed during a simulation) is normalized and the number of epochs in order to arrive at optimal learning rates that are as independent of the system as possible. Since they are only constant factors during training, these do, however, have no influence on the system apart from rescaling the learning rate.

Finally, for molecular systems, there are some further demands towards the loss: Translations and rotations lead, in the naive definition of Eq. (15) to a major decrease in L, whereas in reality the physics of the system has not changed. To circumvent this, there are two possible adaptations: Either only parameters of the integrator which do not (directly) lead to translations (such as Δtᵢ or n) are learned or one restricts the loss to optimize for translational invariant objectives (such as using the jump in relative distances). In this work, the first approach will be followed.

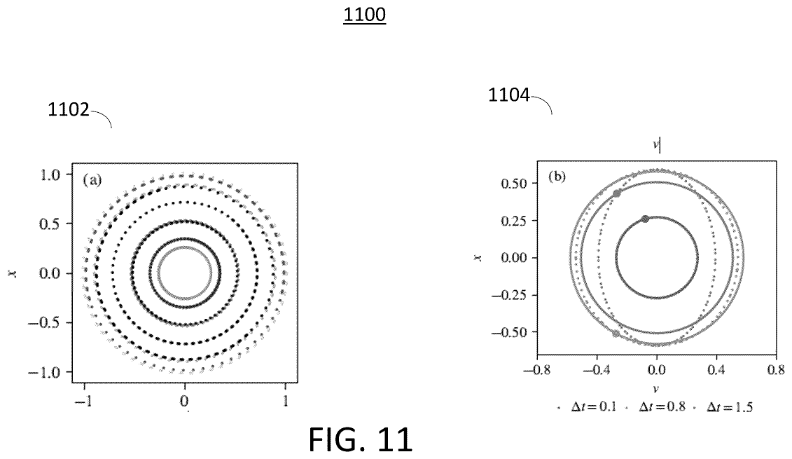

Graph 1102 of FIG. 11 depicts example trajectories for x and v of the one-dimensional harmonic oscillator for Δt=0.1 at T=0.5 as obtained from HMC with n=100. Graph 1104 of FIG. 11 depicts an influence of the choice of Δt on the simulated (shadow) Hamiltonian using otherwise the same parameters as in graph 1102. The solid lines in the same color as the data points correspond to the analytically expected trajectories. The big dots symbolize the starting point of the trajectory, which sets the expected energy level.

Firstly the approach is validated for a simple test-system, the one dimensional harmonic oscillator, which allows to study the effects of HMC in more detail and visualize some open points of the HMC approach. Following this, a physically more realistic system is studied, alanine dipeptide.

The analysis starts by considering one of the simplest physical systems: The one-dimensional harmonic oscillator, which can have physical interpretation for example as a pendulum or a spring fixed at the origin at one end with a single mass attached at the other. The goal is to understand basic principles of HMC and be able to visualize what is happening for this system. The full Hamiltonian of the harmonic oscillator with mass m=1 and spring constant k=1 is defined as:

$$ℋ = U ( x ) + K ( v ) = 0.5 ( {x}^{2} + {v}^{2} ) , \qquad ( 18 )$$

where x is the position and v is the velocity of the mass. The goal for this exemplary system is to simulate it at a fixed temperature in the canonical ensemble, for which T=0.5 is chosen. The general recipe of HMC is presented below.

In graph 1102 of FIG. 11 a few sample trajectories of the harmonic oscillator are visualized for Δt=0.1 and n=100 integration steps obtained from HMC. The trajectories form (near perfect) circles, where the radius is given by energy conservation of the Hamiltonian. The different radii of the circles can be understood since at every new iteration the velocity is picked from the Maxwell-Boltzmann distribution, setting a different level of the total energy. With these parameters, the total energy is nearly perfectly conserved, leading to acceptance rates close to 100%. It is well known that it is not ideal to have such high acceptance rates, since phase space can be much more effectively be explored when using a larger Δt, see IIC for a more detailed discussion. In principle, increasing Δt for fixed number of integration steps n only changes how fast the phase-space is sampled/explored, i.e., how fast the “circle” is examined. There are, however, a couple of interesting effects: It is well known in the literature, that when using a finite time step, one only simulates the so called shadow Hamiltonian and not the true Hamiltonian. The difference between these two Hamiltonians can readily be observed for the harmonic oscillator, for which example trajectories (obtained using the velocity Verlet integrator defined in Eqs.(5)) are plotted for different Δt in graph 1104 of FIG. 11 as would be used to obtain a single HMC proposal. While for Δt=0.1, the obtained trajectory plotted as dots is (nearly) forming a perfect circle at least on the scale shown here, for Δt=0.8 and 1.5 fs the trajectory clearly forms an ellipse with eccentricity e>0. As solid lines in the same color as the data points, the trajectories which should theoretically have been simulated are also drawn, simply following the energy conservation prescribed by the Hamiltonian (18). These true trajectories start from the initial point of the trajectory marked by the big dot in the same color. It can be clearly seen that the points deviate more from the true circle for increasing Δt. Whenever one observes points on the inside of the circle, this corresponds to a lower total energy and thus acceptance of 100%, whereas points outside the circle have larger total energy and are not always accepted. While one would naively expect that these effects should cancel out since one often starts from a new position, resulting in an average acceptance rate in the course of a full HMC simulation, this is not the case. Since the first point of the phase-space sets the initial energy level (which in turn sets the circle) and one always moves along the trajectory the same distance (given by nΔt) in either forward or backward direction, one sees a periodic behavior of the acceptance rate given by the “deviation from the circle” in FIG. 1.

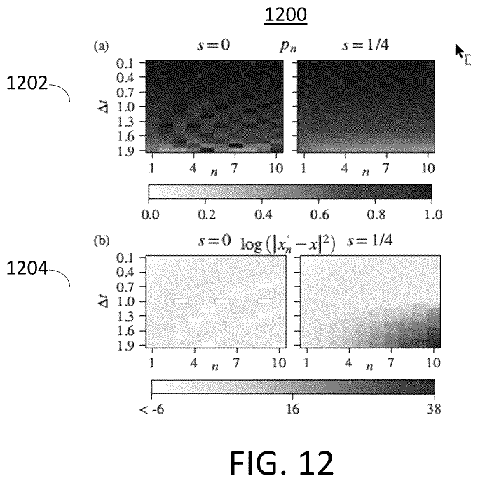

Heatmap 1202 of FIG. 12 depicts acceptance p and heatmap 1204 of FIG. 12 depicts logarithm of squared jump

$${( {x}_{n}^{′} - x )}^{2}$$

as a function of timestep Δt and number of integration steps n for the one-dimensional harmonic oscillator at T=0.5. Shown are in both cases the results for the not jittered (s=0) and jittered (s=¼) time step Δt.

The advantage of jittering the time step Δt on the dynamics of the harmonic oscillator are investigated as an approach to avoid recurring patterns in the simulation. Heatmap 1202 shows a heatmap of the acceptance rate pₙ as a function of time step Δt and number of integration steps n for the harmonic oscillator, measured after the system is equilibrated. Without jittering of the time step Δt, i.e., by setting s=0 in Eq. (8) several minima/maxima are observed in the surface plot, corresponding to small/large acceptance rates. They follow a pattern, which can exactly be explained by the deviations from the true trajectories discussed in graph 1104 of FIG. 11. When introducing jittering on the time step, as shown in the same plot where used s=¼ is used, these minima/maxima vanish and one observes as a function of Δt a smooth decay of the acceptance rate pₙ. This trend is also there in the non-jittered simulation, but less visible due to the overlay with the many minima/maxima. This highlights the problem of using a simple criterion of fixed target acceptance rate, as often done when tuning the parameters of HMC. Without jittering, one would pick, depending on the starting parameters, any pair of Δt and n having the desired value of acceptance rate, which does not need to correspond to a small autocorrelation time. For the jittered simulations, one would pick a fixed Δt as local minima/maxima are smoothed out, but without any ability to distinguish between the influence of n on the performance. As will be seen later, this does not correlate well with the autocorrelation times of the potential energy.

A similar behavior of multiple local minima/maxima can also be observed for the squared jump distance, which is presented in heatmap 1204 of FIG. 12. Here, the plot is logarithmic, since the differences in the jump are quite large for some parameter configurations. Without jittering (s=0), several minima/maxima are observed in the surface plot, whereas with jittering s=¼ this is not seen. With jittering, however, finds that the squared jump distance can become huge for the larger Δt and n region, which can be explained by the occasional “breaking” of simulations at large Δt where self-enforcing effects lead to explosions of the values of the position and velocity. This is a well-known effect when choosing very large time steps and are typically rejected by the Metropolis-Hastings criterion due to a very large potential energy.

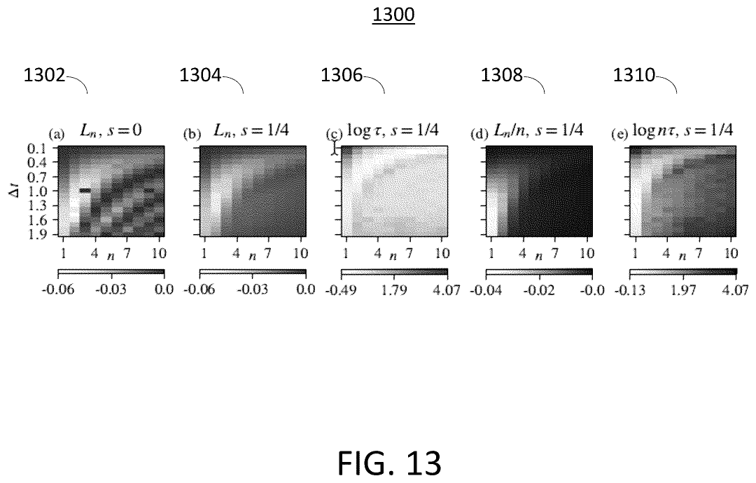

Heatmaps 1302 and 1304 of FIG. 13 show the loss Lₙ of Eq. (15) recorded during the HMC run (without learning, i.e., only showing the obtained values for the optimization target). The general observation of multiple minima/maxima for the separate acceptance rate and squared jump distance in heatmaps 1202 and 1204 of FIG. 12 also carries over to the loss (correlated expectation value of both) without jitter, see heatmap 1302 of FIG. 13. With jitter, as shown in heat map 1304 of FIG. 13, the loss loses this property, and one (clear) global minimum emerges at Δt≈1.3 and n≈2. The goal is that the loss serves as a local proxy for the autocorrelation times of the observables, where the focus is on the correlations of the potential energy as a placeholder for many interesting properties of the system. While not optimally, the loss agrees generally well with the (logarithms of the) autocorrelation times for the potential energy presented in heatmap 1306 FIG. 13. The region with lower loss appears to be shifted relatively towards higher Δt, which however is not as detrimental as smaller Δt. Finally, however, the interested is in the performance per computing effort. For this, Lₙ/n in is plotted in heatmap 1308 of FIG. 13, which shifts the minimum towards smaller n. The global optimum for this system at T=0.5 is somewhere around Δt≈1.75 and n≈1. This is also reconfirmed for the autocorrelation time nτ, measured in terms of the computational effort. Here, it also observe that the minima shift towards smaller n, although not as strongly as for the loss. This is due to the relative difference in the amplitude between minimum/maximum for the loss and autocorrelation time.

After having established a good correlation between the loss and the autocorrelation time of the potential energy for this system, the focus shifts to learning the optimal parameters via the fully differentiable framework, implemented in pyTorch. The Adam optimizer is used with learning rate ϵ=0.01 with otherwise default parameters from pyTorch. Before performing on optimization step of Δt and n, 10 proposals are performed in order to average out the resulting gradients, setting the epoch length. The system is initialized for different Δ₀t and the attention weights Cₙ are picked uniformly from zero to one (this means the mean value of the number of steps n is initially ≈N/2 and allows for “information” from all integration steps).

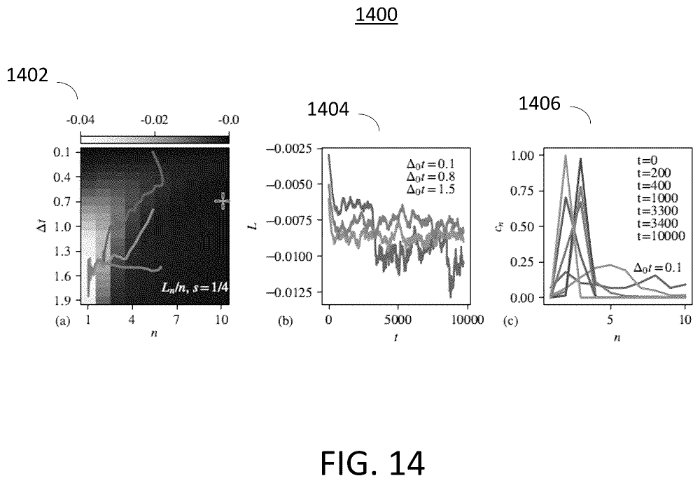

In graph 1402 of FIG. 14 the loss surface of Lₙ/n as presented in heatmap 1308 of FIG. 13 is shown, but now includes sample learning trajectories for different initial values of Δ₀t with the Cₙ randomly initialized as discussed before. The mean values of the time step and number of integration steps are plotted, i.e., Δt and

$$\overline{n} = {\sum }_{i = 1}^{N} n {c}_{n} .$$

It is evident from the visualization of the trajectories that, independent of initial parameter, all curves move towards the region with smaller loss values. This is reinforced by the recorded loss values during training, which a function of training epoch is plotted in graph 1404 of FIG. 14. Since the data for the recorded loss is very noisy due to the small batch size and few degrees of freedoms of the system leading to little self-averaging, a running average is calculated over 300 epochs to visualize the training. Especially for Δ₀t=0.1 some jumps in the loss are visible, mainly around t=3000. This corresponds to the “jump” from a big weight at n=3 to n=2, which can also be appreciated from graph 1406 of FIG. 14, where the attention weights cₙ for different epochs are shown. Initially, at t=0, the weights are nearly uniform, where than for early times at t=200 a small peak forms around n=5. As time progresses, there is a large weight on n=3 which then in the times t pan from t≈1000 shifts towards n=2 at t≈3400. At the final training epoch, the weight is completely on n=2. These observations are consistent with what is seen in graph 1402 of FIG. 14 as movements on the loss surface for Δ₀t=0.1.

Having a good correspondence between loss and the autocorrelation of the potential energy allows us to effectively learn good parameters of HMC for this system via the fully differential framework. Next, a bigger molecular system with more intricate interactions will be considered.

Heatmaps 1302 and 1304 of FIG. 13 show the loss L_{N} as a function of Δt and n for (a) no jittering (s=0) and (b) with jittering (s=¼). In heatmap 1306, the logarithm of the autocorrelation time extracted from the time series of the potential energy is plotted. The region of desired small correlation times correspond reasonable well to the region where the loss is minimized, as shown in heatmap 1304. Heatmap 1308 of FIG. 13 shows the same data as in heatmap 1304, but the loss is rescaled with the computational effort Lₙ/n. Finally, in heatmap 1310 it is shown that the logarithm of the autocorrelation time, but in units of the computational effort nr. The logarithm is chosen for the autocorrelation time to highlight the differences, as these are much larger than in the other plots for the losses.

Graph 1402 of FIG. 4 depicts loss surface as a function of Δt and n. On top, three example trajectories showing the expectations values of Δt and n during learning for three initial values of the time step Δ₀t. Graph 1404 of FIG. 14 depicts the corresponding loss as a function of epochs t for the curves shown in graph 1402. Graph 1406 of FIG. 14 depicts attention weights cₙ for Δ₀t=0.1 for different epochs indicated in the legend.

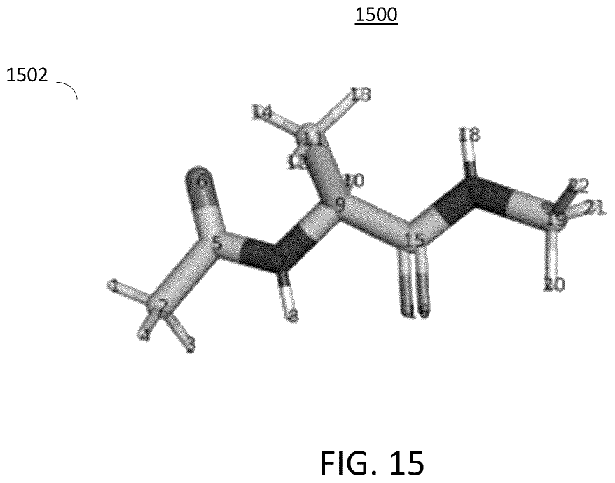

FIG. 15 depicts a graphical representation of alanine dipeptide, where the atoms are marked by their index. The elements 1, 3, 4, 8, 12, 13, 14, 18, 20, 21, and 22 depict hydrogen, elements 2, 5, 9, 11, 15, and 19 depict carbon, elements 6 and 16 represents oxygen, and elements 7 and 17 depict nitrogen.

Alanine dipeptide has proven itself as the most common protein to test novel algorithms, which is why it is investigated in the following. This system is simulated in vacuum, thus this protein has 66 degrees-of-freedom (22 atoms in d=3 spatial dimensions) with some interaction between atoms being bonded, as drawn schematically in FIG. 5. As force-field, Amber-19ffSB is used, for which the implementation from TorchMD is adapted for the purposes. In the original implementation, the gradients calculated by automatic differentiation needed to make this problem fully-differentiable are not preserved. The code is adapted to preserve those. A description on the functional form of the potential energy part of the Hamiltonian is provided in Tian. The temperature is set to T=300 K and free boundary conditions are employed. For the HMC simulations, it is found that jittering with 25% can lead to non-stable simulations for larger Δt, which is why here a jitter is used with 10% relative variance, i.e., s=0.1. The autocorrelation times were calculated with fixed parameters after equilibration and obtained from time series with 2×10⁵ MC proposals.

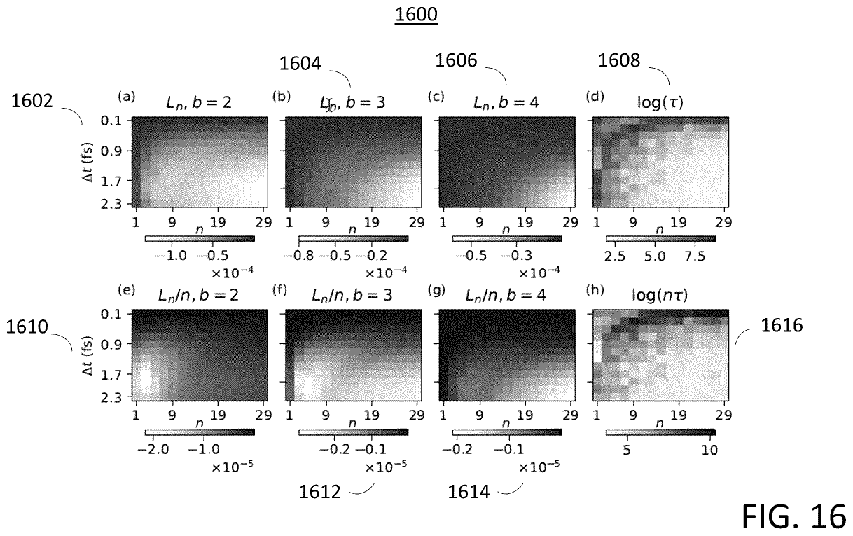

The investigating is begun by correlation between the loss and the autocorrelation time of the potential energy. In heatmaps 1602, 1604, and 1606 of FIG. 16 the loss surfaces for different choices of b in the definition of the loss of Eq. (15) is plotted. A larger value of b promotes big jumps and gives less importance to small jumps, that is, big moves are more important. In all cases, jittering with 10% relative variance is used, resulting in smooth loss surfaces. The surface has been checked to have multiple minima/maxima when no jittering is done, reiterating the importance of its inclusion. In heatmap 1608 of FIG. 16, the corresponding autocorrelation time based on the potential energy is shown. This approach of adapting the loss function is similar in spirit to the one presented in Levy, although the influence was not investigated in detail there. They chose to include a reciprocal term with a positive sign, i.e., the wanted to actively penalize small jumps in the coordinates. The loss proposed in Levy is empirically checked, but suitable parameters for the free parameter k which corresponded to a better match are not found.

In some embodiments, the common definition of a squared jump distance (b=2) in heatmap 1602 of FIG. 16 is found to not correlate well with the actual observed autocorrelation times. The region having a small loss is very large, going down to small number of integrations steps n≈3 for Δt=2.3 fs, whereas for the autocorrelation times the region of the minimum starts around n≈9. As discussed before, there can be several reasons for this mismatch. On the one hand the limitation of optimizing for the lag-1 autocorrelation (made necessary to have a fast converging measure) is a potential source of mismatch and on the other hand the focus on a different observable can introduce problems. Thus, it comes as no big surprise that other definitions of the loss can provide a better correspondence. In heatmaps 1604 and 1606 empirically tests are conducted to determine what happens to the loss surface for b=3 respectively b=4. It is found that for b=3, the minimum region of the loss shifts towards the right (larger number of integration steps n), as expected. The correspondence between the loss surface and the autocorrelation times is much better. For b=4, the minimum of the loss-surface appears still to align well with the autocorrelation time, however, it appears slightly too much favored towards large n. This impression, however, changes once the loss per computational cost Lₙ/n is considered, which is plotted in heatmaps 1610, 1612, and 1614. Due to the small differences in amplitude between the maximal and minimal loss values when compared to the differences between autocorrelation times, the division by n massively shifts the loss towards smaller n, which is not reflected in the autocorrelation time. As for the harmonic oscillator, a less pronounced shift is observed towards smaller n for the autocorrelation time in units of computational effort nτ plotted in (h). From these plots, it is found that the loss for b=4 in heatmap 1616 provides a good correspondence, so that this value will be used for the following analysis.

FIG. 16 depicts the influence of the parameter b in the loss of Eq. (15) on the loss for alanine dipeptide for b=2 in heatmap 1602, b=3 in heatmap 1604, b=4 in heatmap 1606 on the loss surface Lₙ as a function of Δt and n is shown. In heatmap 1610 the corresponding logarithm of the autocorrelation time of the potential energy is presented. Heatmaps 1610, 1612, and 1614 show the corresponding plots of the loss per computational effort Lₙ/n, and heatmap 1616 shows the logarithm of the autocorrelation time in terms of computational effort nτ.

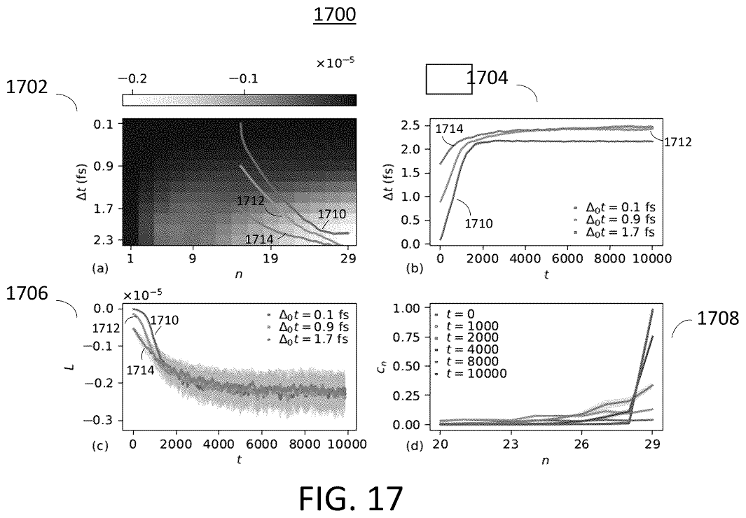

In some embodiments, the focus is now on the parameters of HMC, following the general outline of the previous discussion for the harmonic oscillator. The learning rate is set to ϵ=0.001 and the other parameters of Adam are kept at the default of pyTorch. 10 proposals are performed before taking one optimization step, which corresponds to training on a batch of 10 observations at each epoch. All results shown are averaged over 5 independent learning trajectories for each initial Δ₀t, where each run was performed using a different random number seed responsible for the initialization of the weights Cₙ, the sampling of velocities, and the acceptance/reject step of HMC. In graph 1702 of FIG. 17 the loss surface per computational effort Lₙ/n for b=4 is presented, where three representative learning trajectories are plotted on for three different initial values of Δ₀t, where the lines can be extracted from graphs 1704 and 1706 of FIG. 17. For example, line 1710 of graphs 1702, 1704, and 1706 are plots for the initial value Δ₀t=0.1 fs, line 1712 of graphs 1702, 1704, and 1706 are plots for the initial value of Δ₀t=0.9 fs, and line 1712 of graphs 1702, 1704, and 1706 are plots for the initial value of Δ₀t=1.7 fs. Plotted are the mean value of both Δt and n. The trajectories are obtained by initially setting Δ₀t=0.1 fs, 0:9 fs, and 1.7 fs respectively, whereas the Cₙ are initialized randomly resulting in a mean value close to N=2. It is seen that all simulations approach a very similar optimum of the loss, which also corresponds to a region where the autocorrelation times are small. Note that the curves assume larger values of Δt than the region for which grid-search of parameters was originally performed, as is also clear from graph 1704 of FIG. 17 where Δt is shown as a function of learning epoch t. The values of Δt should be seen in the context of the ones used for classical MD simulations in the canonical ensemble. There, in order to capture the fastest motions and to guarantee stable simulations, a time step of 0.5 fs is typically used for this system (there are approaches that restrict the motion of Hydrogens, allowing via this trade-off a larger time step). The time step Δt≈2.5 found as optimal allows for a nearly five-fold faster simulation, although of course due to the acceptance/reject step of MC some trajectories are rejected. In addition, MC guarantees the exact sampling of the true Hamiltonian since there are no effects due to the discretization of the time step. It is interesting to note that the values of the acceptance rate around the optimal region are in the range from 50% to 60% and by this somewhat smaller than the predicted ideal value of ≈65%, although still compatible.

Graph 1706 of FIG. 17 shows the value of the loss L as a function of training epoch t. All simulations have similar behaving loss curve (with some differences during the initial training), which all arrive at very similar loss values. This is although the values of Δt are quite different, they cannot be distinguished by the definition of the loss. For the start with Δ₀t=0.9 fs as shown by line 1712, the weights given to each integration step for some selected epochs during training in graphs 1708 of FIG. 17 are also plotted. In some embodiments, it is found that starting from a random initialization giving every layer roughly the same weight, the weights move towards larger n quite fast, resulting on a large weight for the maximally considered integration step N=29 for late training times t. There is some interplay between the time step Δt and the number of integration steps n, as for a given (smaller) Δt the optimum of n does not need to coincide with the optimum for a different n and thus the optimization due to the differential set-up shifts its respective optimum. This, potentially, can influence the training, but obstacles in this regard were not observed.

Finally, the focus shifts towards learning not only the standard parameters of HMC, but also a version which has more parameters. A time step is considered which is atom index dependent, i.e., not only is there a single Δt for all atoms, but rather a different Δtᵢ per atom index i.

TABLE I
Autocorrelation times τ for different initial
Δ₀t for atom based timesteps and global timesteps.
In the brackets the error of the mean is noted.

| Δ₀t | 0.1 fs | 0.9 fs | 1.7 fs |
|---|---|---|---|
| τ for atom based Δt | 12.7 (2.6) | 7.5 (9) | 7.5 (1) |
| τ for global Δt | 12.1 (1.8) | 10.0 (1.0) | (1.3) |

In Table I the autocorrelation times for the potential energy are presented. In some embodiments, it is found that using atom based Δtᵢ, the autocorrelation times for Δ₀t=0.9 fs and 1.7 fs are roughly 25% lower compared to their counterpart having a global Δt. Using atom based Δtᵢ has only very little influence on the resulting wall-clock runtime after training, so that incorporating them in practice simply results in the reported speed-up without additional cost. For 0.1 fs, the autocorrelation times using atom based time steps or a global time steps are comparable, which is not surprising since for this case the loss values are also comparable.

As a reminder: there are 22 atoms in alanine dipeptide which means that there are now 22 different parameters for the time step to optimize. For the numbering of the atoms, refer to FIG. 15. Heuristic approaches not based on gradients would most likely be much less efficient at the optimization of these many parameters, which highlights the importance of the fully differentiable approach.

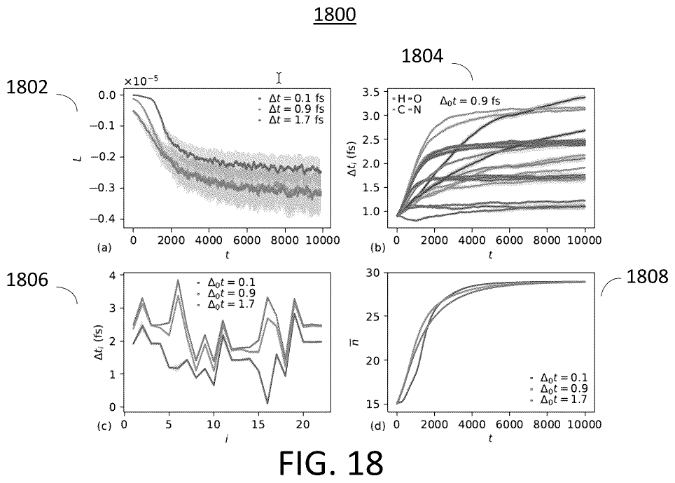

Since the parameter-space is now of much higher dimension, the loss-surface cannot any longer be explored by a grid-search or even easily visualized, which is why marginalized observables are only relied on. In some embodiments, the overall optimization results in smaller loss values, as shown in FIG. 18, as is expected for a more parameterized version of the integrator. Compared to only having a single time-step Δt, a loss value is found after training of about L≈−0.3×10⁻⁵ (Graph 1802 of FIG. 18 is compared to L≈−0.2×10⁻⁵ graph 1706 of FIG. 17), at least for starts with Δ₀t=0.9 fs and 1.7 fs.

Graph 1702 of FIG. 17 depicts loss per computational effort Lₙ/n as a function of Δt and n. Drawn are also three example trajectories obtained during training for initial Δ₀t=0.1 fs using line 1710, 0.7 fs using line 1712, and 1.7 fs using line 1714. Graph 1704 of FIG. 17 shows the value of Δt as a function of the learning epoch t for the three initial conditions. Graph 1706 of FIG. 17 shows the corresponding loss for these three initial conditions as a function of training epoch t. Graph 1708 of FIG. 17 displays the weights cₙ as a function of the corresponding timestep n for different training epochs t as mentioned in the legend, where the focus is on the region of n≥20. In all graphs 1704-1708, the shaded regions in the plot correspond to the error of the mean obtained by averaging over 5 independent runs.

In graph 1804 of FIG. 18 the atom based time steps Δtᵢ are plotted as a function of epoch t for initial Δ₀t=0.9. It is found that the time step for some atom index i is greatly improved relative to others, which corresponds to a larger time step of these atoms. Also, the absolute value is much larger than the average value obtained when optimizing only the “global” time step Δt. Graph 1806 of FIG. 18 shows the final values of Δtᵢ after the learning, which highlights that, at least for the initial values of Δ₀t=0.9 fs and 1.7 fs, a very similar behavior of Δtᵢ is achieved, which is an indicator that there is a local minimum of the loss for the these values of Δtᵢ. There is up to a 3.5 fold difference between the largest and smallest time step, highlighting the differences in the ideal parameters. For Δ₀t=0.1 fs, many signatures as for the other two initial starting parameters remain, i.e., for many atoms the values of Δtᵢ follow the same trend as observed for Δ₀t=0.9 fs and 1.7 fs, although it is unclear whether they would converge to the exactly same value in the long run using local gradient based optimizers. For all initial values of Δ₀t, it is found that for the number of integration steps n, n=29 is approached, i.e., the currently maximal allowed number of integration steps, as shown in graph 1808 of FIG. 18.

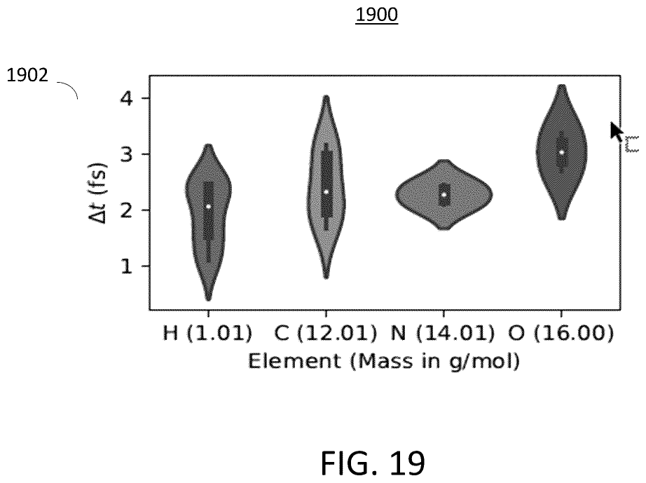

To gain some physical insight into the obtained values for Δtᵢ, the distribution of them are plotted in FIG. 19 for the different atom types of alanine dipeptide, where the values of Δtᵢ are obtained after training with Δ₀t=0.9 fs. The elements are ordered after their mass, showing some positive correlation between the mass of the atom and the ideal time step Δtᵢ. The distribution, however, is very broad in many cases. This implies that this is indeed not a simple property of atom type only, but most likely determined by its local neighborhood and the temperature.

Graph 1802 of FIG. 18 depicts Loss L as a function of epoch t for different initial values of Δ₀t. Graph 1804 of FIG. 18 depicts the individual Δtᵢ per atom for the initial value of Δt=0.9 fs, where the different lines correspond to the plots of elements hydrogen, oxygen, carbon and nitrogen. In graph 1806 the final learned value of Δtᵢ after training is shown for the different initial values of Δt. Finally, graph 1808 shows the mean value of integration steps n as a function of learning epoch t.

FIG. 19 provides a violin plot for the distribution of the time step Δt for the four different atom types present in alanine dipeptide, taken for the start with Δ₀t=0.9 fs. The elements are sorted according to their atom weight, which is mentioned in the brackets up to two digits.

A framework is presented which allows for the gradient-based tuning of Hamiltonian Monte Carlo. Its capabilities are demonstrated for the one dimensional harmonic oscillator and alanine dipeptide simulated at constant temperature. It is found that, in both systems, the set-up allows for the optimization of the parameters of Hamiltonian Monte Carlo, leading to fast simulations and low values of the autocorrelation time.

Further, the parameters of the integrator for alanine dipeptide are extended by introducing time steps that depend on the atom index. In some embodiments, this can lead to lower loss values as compared to using a global time step, which is also reflected in a lower autocorrelation time.

Other definitions of the loss can also be used and may provide an even stronger correlation with the autocorrelation times of the potential energy. Another possibility to further speed up the simulations is the combination with hand-crafted Monte Carlo updates, which can be used in conjunction with the optimized integration to propose changes to the system. For example the pivot-move has been proven to be exceptionally efficient in the high-temperature phase to decorrelate samples.

While the functional form of the integrator is physically motivated and can be considered a good starting point for optimizations, the integration in general can be performed by an arbitrary function, which does not even need to be volume-preserving. In this regard, a “data driven” integrator would be interesting, i.e., it would be interesting to investigate much more heavily parameterized versions of the integrator. Although here, in contrast to the method, the additional cost has to be considered, which can eat up some advantages of a more complicated form. These integrators could then either just have (non-volume) preserving parameters which are tuned as part of the computation graph, but independent of the configuration or an architecture can be chosen based on neural networks with explicit input of the systems phase-configuration, i.e., in some sense a completely different functional form for the integrator can be created. Finally, there is also potential to combine this with machine learning methods operating on graph structured data, for instance with recent equivariant network architectures.

### REFERENCES

T. Schlick and S. Portillo-Ledesma, Biomolecular modeling thrives in the age of technology, Nature computational science 1, 321 (2021).
S. A. Hollingsworth and R. 0. Dror, Molecular dynamics simulation for all, Neuron 99, 1129 (2018).
A. Vitalis and R. V. Pappu, Methods for monte carlo simulations of biomacromolecules, Annual reports in computational chemistry 5, 49 (2009).
D. Frenkel and B. Smit, Understanding molecular simulation: from algorithms to applications, Vol. 1, Elsevier (2001).
P. H. Hiinenberger, Thermostat algorithms for molecular dynamics simulations, Adv. Comput. Sim., 105 (2005).
M. P. Allen and D. J. Tildesley, Computer simulation of liquids, Oxford university press (2017).
R. M. Neal et al., Mcmc using Hamiltonian dynamics, Handbook of Markov chain Monte Carlo 2, 2 (2011).
R. M. Neal, Bayesian learning for neural networks, Vol. 118, Springer Science & Business Media (2012).
S. Duane, A. D. Kennedy, B. J. Pendleton, and D. Roweth, Hybrid Monte Carlo, Physics letters B 195, 216 (1987).
S. Doerr, M. Majewski, A. Perez, A. Kramer, C. Clementi, F. Noe, T. Giorgino, and G. De Fabritiis, Torchmd: A deep learning framework for molecular simulations, Journal of chemical theory and computation 17, 2355 (2021).
D. Levy, M. D. Hoffman, and J. Sohl-Dickstein, Generalizing Hamiltonian Monte Carlo with neural networks, arXiv 10.48550/arXiv.1711.09268 (2021).
M. E. Newman and G. T. Barkema, Monte Carlo methods in statistical physics, Clarendon Press (1999).
W. Janke, Monte Carlo simulations in statistical physics—from basic principles to advanced applications, in Order, Disorder and Criticality: Advance Problems of Phase Transition Theory Volume 3 (World Scientific) pp. 93-166 (2013).
N. Metropolis, A. W. Rosenbluth, M. N. Rosenbluth, A. H. Teller, and E. Teller, Equation of state calculations by fast computing machines, The journal of chemical physics 21, 1087 (1953).
W. K. Hastings, Monte Carlo sampling methods using Markov chains and their applications, Biometrika 57, 97 (1970).
J. Deutsch, Long range moves for high density polymer simulations, J. Chem. Phys, 8849 (1947).
H. Christiansen, S. Majumder, and W. Janke, Phase ordering kinetics of the long-range ising model, Physical Review E 99, 011301 (2019).
H. Christiansen, S Majumder, M. Henkel, and W. Janke, Aging in the long-range ising model, Physical Review Letters 125, 180601 (2020).
T. W. Kibble, Some implications of a cosmological phase transition, Physics Reports 67, 183 (1980).
W. H. Zurek, Cosmological experiments in condensed matter systems, Physics Reports 276, 177 (1996).
J. Song, S. Zhao, and S. Ermon, A-nice-mc: Adversarial training for mcmc, Advances in Neural Information Processing Systems 30 (2021).
S. Liu and S. Sun, Adversarially training mcmc with non-volume-preserving-flows, Entropy 24, 415 (2022).
L. Dinh, J. Sohl-Dickstein, and S. Bengio, Density estimation using Real NVP, arXiv preprint arXiv:1605.08803 (2016).
T. Hastie, R. Tibshirani, J. H. Friedman, and J. H. Friedman, The elements of statistical learning: data mining, inference and prediction, Vol. 2, Springer (2009).
F. Rosenblatt, Principles of neurodynamics, perceptions and the theory of brain mechanisms, Tech Rep. Cornell Aeronautical Lab Inc. Buffalo NY (1961).
D. E. Rumelhart, G. E. Hinton, and R. J. Williams, Learning internal representations by error propagation, Tech. Rep. California Univ San Diego La Jolla Inst for Cognitive Science (1985)
M. Lal and D. Spencer, “Monte Carlo” computer simulation of chain molecules: V. flexibility of n-alkane molecules, Molecular physics 26, 1 (1973).
N. Madras and A. D. Sokal, The pivot algorithm: a highly efficient Monte Carlo method for the self-avoiding walk, Journal of Statistical Physics 50, 109 (1988).
S. A. Hollingsworth and R. O. Dror, “Molecular dynamics simulation for all,” Neuron 99, 1129-1143 (2018).
A. Vitalis and R. V. Pappu, “Methods for monte carlo simulations of biomacromolecules,” Ann. Rep. Comput. Chem. 5, 49-76 (2009).
D. Frenkel and B. Smit, Understanding molecular simulation: from algorithms to applications, Vol. 1 Elsevier, Amsterdam (2001).
P. H. Hünenberger, Advances in Polymer Science, edited by C. Holm and K. Kremer, Vol. 174 Chap. Thermostat algorithms for molecular dynamics simulations, pp. 105-149, Springer, Berlin, Heidelberg (2005).
M. P. Allen and D. J. Tildesley, Computer simulation of liquids, Oxford University Press, Oxford (2017).
R. M. Neal, Handbook of Markov Chain Monte Carlo, edited by S. Brooks, A. Gelman, G. Jones, and X.-L. Meng, 11, Chap. MCMC using Hamiltonian dynamics, p. 2, Chapman and Hall/CRC, New York, (2011).
R. M. Neal, Bayesian learning for neural networks, Lecture Notes in Statistics, Vol. 118, Springer, New York, (2012).
M. Betancourt, “A conceptual introduction to Hamiltonian Monte Carlo,” arXiv:1701.02434 (2017).
S. Duane, A. D. Kennedy, B. J. Pendleton, and D. Roweth, “Hybrid Monte Carlo,” Phys. Lett. B 195, 216-222 (1987).
F. Rosenblatt, “Principles of neurodynamics. perceptrons and the theory of brain mechanisms,” Tech. Rep. Cornell Aeronautical Lab, Buffalo (1961).
D. E. Rumelhart and J. L. McClelland, “Learning internal representations by error propagation,” in Parallel Distributed Processing: Explorations in the Microstructure of Cognition: Foundations, MIT Press, Cambridge, pp. 318-362 (1987).
J. Hermans, “The amino acid dipeptide: Small but still influential after 50 years,” Proc. Nat. Acad. Sci. 108, 3095-3096 (2011).
M. E. Newman and G. T. Barkema, Monte Carlo methods in statistical physics, Clarendon Press (1999).
W. Janke, “Monte Carlo simulations in statistical physics: From basic principles to advanced applications,” in Order, Disorder and Criticality, Advanced Problems of Phase Transition Theory, Vol. 3, edited by Y. Holovatch, pp. 93-166, World Scientific, Singapore, (2013).
N. Metropolis, A. W. Rosenbluth, M. N. Rosenbluth, A. H. Teller, and E. Teller, “Equation of state calculations by fast computing machines,” J. Chem. Phys. 21, 1087-1092 (1953).
W. K. Hastings, “Monte Carlo sampling methods using Markov chains and their applications,” Biometrika 57, 97-109 (1970).
J. Deutsch, “Long range moves for high density polymer simulations,” J. Chem. Phys, 8849-8854 (1997).
H. Christiansen, S. Majumder, and W. Janke, “Phase ordering kinetics of the long-range Ising model,” Phys. Rev. E 99, 011301 (2019).
H. Christiansen, S. Majumder, M. Henkel, and W. Janke, “Aging in the long-range Ising model,” Phys. Rev. Lett. 125, 180601 (2020).
T. W. Kibble, “Some implications of a cosmological phase transition,” Phys. Rep. 67, 183-199 (1980).
W. H. Zurek, “Cosmological experiments in condensed matter systems,” Phys. Rep. 276, 177-221 (1996).
W. C. Swope, H. C. Andersen, P. H. Berens, and K. R. Wilson, “A computer simulation method for the calculation of equilibrium constants for the formation of physical clusters of molecules: Application to small water clusters,” J. Chem. Phys. 76, 637-649 (1982).
S. Prokhorenko, K. Kalke, Y. Nahas, and L. Bellaiche, “Large scale hybrid Monte Carlo simulations for structure and property prediction,” npj Comput. Mat. 4, 80 (2018).
J. Hu, A. Ma, and A. R. Dinner, “Monte Carlo simulations of biomolecules: The MC module in CHARMM,” J. Comput. Chem. 27, 203-216 (2006).
M. Fernández-Pendás, B. Escribano, T. Radivojevic, and E. Akhmatskaya, “Constant pressure hybrid Monte Carlo simulations in GROMACS,” J. Mol. Model. 20, 1-10 (2014).
J. Chodera, A. Rizzi, L. Naden, K. Beauchamp, P. Grinaway, J. Fass, A. Wade, B. Rustenburg, I. Pulido, G. A. Ross, M. Henry, A. Kramer, H. B. Macdonald, J. Rodríguez-Guerra, I. Zhang, A. Simmonett, D. W. Swenson, M. J. Williamson, J. Fennick, S. Roet, S. Boothroyd, A. Silveira, and D. Rufa, “choderalab/openmmtools: 0.21.5,” (2022).
S. Toxvaerd, O. J. Heilmann, and J. C. Dyre, “Energy conservation in molecular dynamics simulations of classical systems,” J. Chem. Phys. 136 (2012).
J. Song, S. Zhao, and S. Ermon, “A-nice-mc: Adversarial training for MCMC,” Adv. Neur. Inf. Proc. Sys. 30 (2017).
S. Liu and S. Sun, “Adversarially training MCMC with non-volume-preserving flows,” Entropy 24, 415 (2022).
A. Beskos, N. Pillai, G. Roberts, J.-M. Sanz-Serna, and A. Stuart, “Optimal tuning of the hybrid Monte Carlo algorithm,” Bernoulli 19, 1501-1534 (2013).
Z. Wang, S. Mohamed, and N. Freitas, “Adaptive Hamiltonian and Riemann manifold Monte Carlo,” Int. Conf. Mach. Learn., 1462-1470 (2013).
M. D. Hoffman and A. Gelman, “The No-U-Turn sampler: adaptively setting path lengths in Hamiltonian Monte Carlo,” J. Mach. Learn. Res. 15, 1593-1623 (2014).
M. Hoffman, A. Radul, and P. Sountsov, “An adaptive-MCMC scheme for setting trajectory lengths in Hamiltonian Monte Carlo,” Int. Conf. Artificial Intel. Stat., 3907-3915 (2021).
M. Girolami and B. Calderhead, “Riemann manifold Langevin and Hamiltonian Monte Carlo methods,” J. Royal Stat. Soc. Ser. B: Stati. Method. 73, 123-214 (2011).
M. D. Parno and Y. M. Marzouk, “Transport map accelerated markov chain monte carlo,” J. Uncert. Quant. 6, 645-682 (2018).
M. Betancourt, “Incomplete reparameterizations and equivalent metrics,” arXiv:1910.09407 (2019).
M. Hoffman, P. Sountsov, J. V. Dillon, I. Langmore, D. Tran, and S. Vasudevan, “Neutra-lizing bad geometry in Hamiltonian monte carlo using neural transport,” arXiv:1903.03704 (2019).
L. Grenioux, A. Durmus, É. Moulines, and M. Gabrié, “On sampling with approximate transport maps,” arXiv:2302.04763 (2023).
J. A. Brofos and R. R. Lederman, “On numerical considerations for riemannian manifold hamiltonian monte carlo,” arXiv:2111.09995 (2021).
T. A. Bojesen, “Policy-guided Monte Carlo: Reinforcement learning Markov Chain dynamics,” Phys. Rev. E 98, 063303 (2018).
E. G. Tabak and E. Vanden-Eijnden, “Density estimation by dual ascent of the log-likelihood,” Comm. Math. Sci. 8, 217-233 (2010).
E. G. Tabak and C. V. Turner, “A family of nonparametric density estimation algorithms,” Comm. Pure Appl. Math. 66, 145-164 (2013).
G. Papamakarios, E. Nalisnick, D. J. Rezende, S. Mohamed, and B. Lakshminarayanan, “Normalizing flows for probabilistic modeling and inference,” J. Mach. Learn. Res. 22, 2617-2680 (2021).
F. Noé, S. Olsson, J. Köhler, and H. Wu, “Boltzmann generators: Sampling equilibrium states of many-body systems with deep learning,” Science 365, eaaw1147 (2019).
M. Gabrid, G. M. Rotskoff, and E. Vanden-Eijnden, “Adaptive Monte Carlo augmented with normalizing flows,” Proc. Nat. Acad. Sci. 119, e2109420119 (2022).
D. Levy, M. D. Hoffman, and J. Sohl-Dickstein, “Generalizing Hamiltonian Monte Carlo with neural networks,” Int. Conf. Learn. Repr. (2018).
A. Paszke, S. Gross, F. Massa, A. Lerer, J. Bradbury, G. Chanan, T. Killeen, Z. Lin, N. Gimelshein, L. Antiga, A. Desmaison, A. Kopf, E. Yang, Z. DeVito, M. Raison, A. Tejani, S. Chilamkurthy, B. Steiner, L. Fang, J. Bai, and S. Chintala, “Py-Torch: An imperative style, high-performance deep learning library,” Adv. Neur. Inf. Proc. Systems, 8024-8035 (2019).
L. B. Rall, Automatic differentiation: Techniques and applications, Lecture Notes in Computer Science (Springer, Berlin, Heidelberg, 1981).
A. Paszke, S. Gross, S. Chintala, G. Chanan, E. Yang, Z. De-Vito, Z. Lin, A. Desmaison, L. Antiga, and A. Lerer, “Automatic differentiation in pyTorch,” Adv. Neur. Inf. Proc. Sys. (2017).
D. P. Kingma and J. Ba, “Adam: A method for stochastic optimization,” Int. Conf. Learn. Repr. (2014).
J. V. Dillon, I. Langmore, D. Tran, E. Brevdo, S. Vasudevan, D. Moore, B. Patton, A. Alemi, M. Hoffman, and R. A. Saurous, “Tensorflow distributions,” arXiv:1711.10604 (2017).
C. J. Geyer, “Practical Markov chain Monte Carlo,” Stat. Sci., 473-483 (1992).
A. Beskos, G. Roberts, and A. Stuart, “Optimal scalings for local Metropolis-Hastings chains on nonproduct targets in high dimensions,” Ann. Appl. Probab. 19, 863-898 (2009).
C. Pasarica and A. Gelman, “Adaptively scaling the Metropolis algorithm using expected squared jumped distance,” Stat. Sinica, 343-364 (2010).
P. Sountsov and M. D. Hoffman, “Focusing on difficult directions for learning HMC trajectory lengths,” arXiv:2110.11576 (2021).
A. Vaswani, N. Shazeer, N. Parmar, J. Uszkoreit, L. Jones, A. N. Gomez, L. Kaiser, and I. Polosukhin, “Attention is all you need,” Adv. Neur. Inf. Proc. Sys. 30 (2017).
R. D. Engle, R. D. Skeel, and M. Drees, “Monitoring energy drift with shadow Hamiltonians,” J. Comput. Phys. 206, 432-452 (2005).
O. A. Zolotov and V. E. Zalizniak, “Accurate energy conservation in molecular dynamics simulation,” Nanosyst. Phys. Chem. Math. 4, 657-669 (2013).
S. Kim, “Time step and shadow Hamiltonian in molecular dynamics simulations,” J. Korean Phys. Soc. 67, 418-422 (2015).
S. Doerr, M. Majewski, A. Perez, A. Kramer, C. Clementi, F. Noe, T. Giorgino, and G. De Fabritiis, “Torchmd: A deep learning framework for molecular simulations,” J. Chem. Theo. Comput. 17, 2355-2363 (2021).
C. Tian, K. Kasavajhala, K. A. Belfon, L. Raguette, H. Huang, A. N. Migues, J. Bickel, Y. Wang, J. Pincay, Q. Wu, et al., “ff19sb: Amino-acid-specific protein backbone parameters trained against quantum mechanics energy surfaces in solution,” J. Chem. Theo. Comput. 16, 528-552 (2019).
M. Lal and D. Spencer, “Monte Carlo computer simulation of chain molecules: V. Flexibility of n-alkane molecules,” Mol. Phys. 26, 1-6 (1973).
N. Madras and A. D. Sokal, “The pivot algorithm: a highly efficient Monte Carlo method for the self-avoiding walk,” J. Stat. Phys. 50, 109-186 (1988).
D. Bacciu, F. Errica, A. Micheli, and M. Podda, “A gentle introduction to deep learning for graphs,” Neur. Net. 129, 203-221 (2020).
M. M. Bronstein, J. Bruna, T. Cohen, and P. Velickovic, “Geometric deep learning: Grids, groups, graphs, geodesics, and gauges,” arXiv:2104.13478 (2021).

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications can be made, by those of ordinary skill in the art, within the scope of the following claims, which can include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

1. A computer-implemented method for accelerating the simulation of a molecular system, the method comprising:
learning a first set of parameters by executing a Hamiltonian Monte Carlo method on the molecular system based on a set of initial conditions;
executing a simulation of the molecular system based on the first set of parameters;
predicting thermodynamics expectation values of the molecular system based on the executed simulation; and
providing the predicted thermodynamics expectation values for a downstream machine learning task.

2. The method of claim 1, wherein the Hamiltonian Monte Carlo method uses a leap-frog integrator including learnable functions realized by neural networks.

3. The method of claim 2, wherein the neural networks are configured such that a computed loss is invariant to translations and rotations of molecules of the molecular system.

4. The method of claim 2, wherein the neural networks are configured such that outputs of the neural network transform equivariantly to rotations of molecules of the molecular system.

5. The method of claim 2, wherein the neural networks are configured to learn atom dependent time steps so as to only learn parameters that cannot lead to translations or rotations.

6. The method of claim 1, wherein learning the first set of parameters includes only considering rescaling parameters that are invariant to a molecular transformation.

7. The method of claim 1, wherein the set of initial conditions comprises a description of the molecular system, functional form of forces and energy, and a second set of parameters for the simulation.

8. The method of claim 7, wherein the description of the molecular system comprises a list of molecules to be investigated, and physical conditions of the molecular system.

9. The method of claim 1, wherein the simulation of the molecular system is executed for a fixed number of compute operations.

10. The method of claim 1, wherein the simulation of the molecular system is executed for a fixed time.

11. The method of claim 1, wherein the predicted thermodynamics expectation values comprise binding affinities for ligand-protein or protein-protein pairs, and the downstream machine learning task simulates ligand-protein or protein-protein interaction for drug discovery in medical AI/healthcare.

12. The method of claim 1, wherein the predicted thermodynamics expectation values comprise material properties such as stability, responses to shear, or responses to solvents, and the downstream machine learning task simulates bio-plastic or plastic thermodynamical and mechanical properties.

13. The method of claim 1, wherein the predicted thermodynamics expectation values comprise binding energies as proxy for efficiency of catalysts used in the molecular system, and the downstream machine learning task simulates small molecules for catalysis for energy storage and production.

14. A computer system programmed for accelerating the simulation of a molecular system, the computer system comprising one or more hardware processors which, alone or in combination, are configured to provide for execution of the following steps:
learning a first set of parameters by executing a Hamiltonian Monte Carlo method on the molecular system based on a set of initial conditions;
executing a simulation of the molecular system based on the first set of parameters for a fixed number of compute operations;
predicting thermodynamics expectation values of the molecular system based on the executed simulation; and
providing the predicted thermodynamics expectation values for a downstream machine learning task.

15. A tangible, non-transitory computer-readable medium for accelerating the simulation of a molecular system having instructions thereon, which, upon being executed by one or more processors, provides for execution of the following steps:
learning a first set of parameters by executing a Hamiltonian Monte Carlo method on the molecular system based on a set of initial conditions;
executing a simulation of the molecular system based on the first set of parameters for a fixed number of compute operations;
predicting thermodynamics expectation values of the molecular system based on the executed simulation; and
providing the predicted thermodynamics expectation values for a downstream machine learning task.
